(19) United States
(12) Patent Application Publication
(10) Pub. No.: US 2015/0074847 A1
(43) Pub. Date: Mar. 12, 2015

(54) ENHANCING PROTEIN STABILITY IN TRANSGENIC PLANTS

(71) Applicant: THE ROCKEFELLER UNIVERSITY, New York, NY (US)

(72) Inventors: Nam-Hai Chua, New York, NY (US); In Cheol Jang, Singapore (SG); Qiwen Niu, Staten Island, NY (US); Shulin Deng, New York, NY (US)

(73) Assignee: THE ROCKEFELLER UNIVERSITY, New York, NY (US)

(21) Appl. No.: 14/391,626

(22) PCT Filed: Mar. 13, 2013
(86) PCT No.: PCT/US13/30953

§ 371 Date: Oct. 9, 2014

Related U.S. Application Data
(60) Provisional application No. 61/623,794, filed on Apr. 13, 2012.

(51) Int. Cl. C12N 15/62; C07K 14/415; C12N 15/82

(52) U.S. Cl. CPC C12N 15/62; C12N 15/8257; C12N 15/8209; C07K 14/415; C07K 2319/00
USPC 800/278; 435/320.1; 435/419; 800/298; 530/377

(57) ABSTRACT

The present invention provides compositions and methods for enhancing protein stability in transgenic plants. The compositions are nucleic acid constructs which encode fusion proteins, fusion proteins, transgenic plant cells and transgenic plants. A fusion protein in accordance with the present invention comprises a protein of interest and a UBA1 or UBA2 domain of an Arabidopsis RAD23 protein. The methods use the nucleic acid constructs to produce fusion proteins in transgenic plant cells or transgenic plants. The fusion proteins have greater stability than the protein of interest and have the same function as the proteins of interest.

### SEQUENCE SUBMISSION

The present application is being filed along with a Sequence Listing in electronic format. The Sequence Listing is entitled 2312129PCTSequenceListing.txt, was created on 8 Mar. 2013 and is 13 kb in size. The information in the electronic format of the Sequence Listing is part of the present application and is incorporated herein by reference in its entirety.

### BACKGROUND OF THE INVENTION

The present invention relates to the field of plant molecular biology, more particularly to gene expression stabilization in transgenic plants.

The publications and other materials used herein to illuminate the background of the invention, and in particular, cases to provide additional details respecting the practice, are incorporated by reference in their entirety for all that they disclose, and for convenience are referenced in the following text by author and date and are listed alphabetically by author in the appended bibliography.

Transgene expression varies widely amongst transgenic plant lines depending on insertion events. It is not unusual to screen many transgenic events just to obtain one or a few lines of the desired transgene expression level. Any technologies that can enhance transgene expression level would therefore be of value to basic plant biology research as well as crop biotechnology.

In the past, optimization of transgene expression has focused primarily on transcription by increasing, promoter strength and enhancing tissue specific expression. For transgenes derived from non-plant sources efforts have been made toward increasing translation efficiency by the use of a viral 5′ non-translated leader, optimization of sequences surrounding the initiation codon and alterations of codon usage to plant-preferred ones (Streatfield, 2007). Recent advances in knowledge on mRNA decapping and degradation pathways (Xu and Chua, 2011) and small RNA-mediated post-transcriptional gene silencing (Mallory and Vaucheret, 2010) have led to the realization of the importance of transcript stability as well. By contrast, relatively little work has been done on how to increase the stability of transgene-derived protein products. Two groups have reported that fusions of coding sequences to the C-terminus of ubiquitin (Ub) can increase protein accumulation (Garbarino et al., 1995; Hondred et al., 1999); however, in these cases, the relative contributions of translation efficiency and protein stability were not determined.

All plant proteins have an intrinsic half-life depending on their function and cellular location. Metabolic enzymes are generally more stable than signaling pathway components as rapid turnover of the latter is part of their regulatory mechanism (Henriques et al., 2009). Most plant signaling pathways terminate in protein factors that regulate transcription of gene networks. The Arabidopsis genome encodes more than 2,000 transcription factor (TF) genes (Mitsuda and Ohme-Takagi, 2009) and their regulatory importance has prompted researchers to use over-expression to investigate TF function and regulatory network as well as to impart new traits (e.g. drought resistance) on transgenic crops. However, several transcription factor genes when over-expressed elicit only a weak phenotype (Xie et al., 2001) or no phenotype at all because transgene expression level was limited not by mRNA levels but by protein stability (Seo et al., 2003; Jang et al., 2005; Jang et al., 2007).

Most plant proteins, other than those residing in organelles such as plastids and mitochondria, are degraded by the ubiquitin/26 proteasome (Ub/proteasome) pathway (Vierstra, 2009). In this pathway, degradation motifs (e.g. PEST sequences) in proteins signal ubiquitination and the ubiquitinated proteins are then escorted to 26S proteasomes for destruction (Fu et al., 2010). Available evidence suggests that ubiquitinated proteins are somehow unfolded at the entrance of the proteasomal particle and become degraded as the unfolded protein passes through the channel of the 26S proteasome (Voges et al., 1999). In theory, any mechanism that can intervene with one or more steps in the Ub/proteasome pathway is expected to prolong protein half-life; however, such an intervention would not be specific and would produce a general effect on protein stability and accumulation, which would be undesirable. To override the instability of a specific protein it is necessary to identify portable amino acid sequences that can confer stability in cis. Such a sequence has been reported by Heessen et al. (2005) who used the ubiquitin-associated (UBA) 2 (UBA2) domain (also referred to herein as sequence) derived from the yeast RAD23 protein to increase stability of a destabilized GFP reporter protein in yeast in which the GFP reporter protein was destabilized by fusing it to ubiquitin. Interestingly, the UBA1 sequence from the same RAD23 protein was found to be inactive (Heessen et al, 2005).

Thus, it is desired to enhance and stabilize transgene expression level in transgenic plants for plant biotechnology.

### SUMMARY OF THE INVENTION

The present invention provides compositions and methods for enhancing protein stability in transgenic plants. The compositions are nucleic acid constructs which encode fusion proteins, fusion proteins, transgenic plant cells and transgenic plants. A fusion protein in accordance with the present invention comprises a protein of interest and a UBA domain as described herein. The methods use the nucleic acid constructs to produce fusion proteins in transgenic plant cells or transgenic plants. The fusion proteins have greater stability than the proteins of interest and have the same function as the proteins of interest.

Thus, in a first aspect, the present invention provides a nucleic acid construct comprising a plant operable promoter operably linked to a nucleic acid encoding a fusion protein. In one embodiment, the nucleic acid comprises a first nucleic acid segment and a second nucleic acid segment. In another embodiment, the first nucleic acid segment is a DNA of interest which encodes a protein of interest. In an additional embodiment, the second nucleic acid segment encodes a UBA domain as described herein. In a further embodiment, the UBA domain is fused to the C-terminus of the protein of interest.

In a second aspect, the present invention provides a fusion protein. In one embodiment, the fusion protein comprises a first protein segment and a second protein segment. In another embodiment, the first protein segment is a protein of interest. In an additional embodiment, the second protein segment is a UBA domain as described herein. In a further embodiment, the UBA domain is fused to the C-terminus of the protein of interest.

In a third aspect, the present invention provides a transgenic plant cell comprising the nucleic acid construct. In one embodiment, the fusion protein is expressed in the transgenic plant cell. In another embodiment, the fusion protein is more stable in the transgenic plant cell than the corresponding protein of interest. In an additional embodiment, the fusion protein has the same function in the transgenic plant cell as the corresponding protein of interest.

In a fourth aspect, the present invention provides a transgenic plant comprising the nucleic acid construct. In one embodiment, the fusion protein is expressed in the transgenic plant. In another embodiment, the fusion protein is more stable in the transgenic plant than the corresponding protein of interest. In an additional embodiment, the fusion protein has the same function in the transgenic plant as the corresponding protein of interest.

In a fifth aspect, the present invention provides a method of enhancing the stability of a protein of interest in a transgenic plant cell or in a transgenic plant. In one embodiment, the method comprises transfecting a plant cell with the nucleic acid construct to produce a transgenic plant cell as described herein. The method further comprises expressing the fusion protein in the transgenic plant cell as described herein. The expressed fusion protein is more stable in the transgenic plant cell than the corresponding protein of interest and has the same function in the transgenic plant cell as the corresponding protein of interest. The method may optionally include preparing a nucleic acid construct encoding a fusion protein as described herein. In another embodiment, the method comprises regenerating a transgenic plant from the transgenic plant cell. In this embodiment, the fusion protein is expressed in the transgenic plant. The expressed fusion protein is more stable in the transgenic plant than the corresponding protein of interest and has the same function in the transgenic plant as the corresponding protein of interest.

### BRIEF DESCRIPTION OF THE FIGURES

FIGS. 1a-1c show that Arabidopsis RAD23 proteins are relatively stable. FIG. 1a: Western blot analysis showing RAD23s (a to d) levels in WT Arabidopsis (Col) with anti-RAD23b (α-RAD23b). FIG. 1b: Western blot analysis showing RAD23s (a to d) levels with or without MG132. Two week-old Arabidopsis plants were treated with or without MG132 (50 μM), which blocks protein degradation. FIG. 1c: Western blot analysis showing changes in RAD23 (a to d) levels after cycloheximide (CHX) treatment. FIGS. 1a-1c: Two week-old Arabidopsis plants were used. Tubulin levels (α-tub) were used as loading controls.

FIGS. 2a-2c show the construction of 6Myc-HFR1-UBA1 and 6Myc-HFR1-UBA2. and analysis. FIG. 2a: Schematic diagrams of 6Myc-HFR1 (M-HFR1), 6Myc-HFR1-UBA1 (M-HFR1-U1), and 6Myc-HFR1-UBA2 (M-HFR1-U2). FIG. 2b: HRF1 transcript levels of transgenic Arabidopsis plants (M-HFR1, M-HFR1-U1, and M-HFR1-U2). Two independent lines (#1 and #2) of each genotype expressing comparable HFR1 transcript levels were selected for this analysis. FIG. 2c: Western blot analysis of HFR1 levels (α-Myc) in transgenic Arabidopsis plants overexpressing M-HFR1, M-HFR1-U1 or M-HFR1-U2 with or without MG132. Lines #1 and #2 of each genotype were analyzed and HFR1 levels were detected by anti-Myc antibodies. Tubulin levels were used as a loading control.

FIGS. 3a-3c show that Arabidopsis HFR1 is stabilized by UBA1/2 fusion. FIG. 3a: HFR1 protein decay is delayed by UBA1 or UBA2 fusion. Two week-old transgenic Arabidopsis plants (M-HFR1, line #2; M-HFR1-U1, line #2; and M-HFR1-U2, line #2) were incubated in liquid MS medium with MG132 (50 μM) for 12 h, washed, and then transferred to fresh MS medium with 100 μM cycloheximide (CHX) and samples were taken at different times as indicated. Proteins were analyzed by western blots as detailed in Example 1. Numbers below each lane indicate relative expression levels. Tubulin levels were used as a loading control. FIG. 3b: Time course of HFR1 protein decay after cycloheximide (CHX) treatment. Values from (a) were analyzed. FIG. 3c: Analysis of hypocotyl length of transgenic Arabidopsis seedlings expressing M-HFR1, M-HFR1-U1, and M-HFR1-U2 under FR light. Two independent lines (#1 and #2) from each genotype were examined. Seedlings were grown for 4 d under FR light (1 μmol m⁻² s⁻¹) on MS media without sucrose. Data were presented as average hypocotyl length±standard deviations (SD; n>40).

FIGS. 4a-4c show the characterization of 3HA-PIF3, 3HA-PIF3-UBA1 and 3HA-PIF3-UBA2. FIG. 4a: Schematic diagrams of 3HA-PIF3 (H-PIF3), 3HA-PIF3-UBA1 (H-PIF3-U1), and 3HA-PIF3-UBA2 (H-PIF3-U2). FIG. 4b: PIF3 transcript levels of transgenic Arabidopsis plants (H-PIF3, H-PIF3-U1, and H-PIF3-U2). Two independent lines (#1 and #2) of each genotype expressing comparable PIF3 transcript levels were selected for this analysis. FIG. 4c: Western blot analysis showing PIF3 levels (α-HA) in transgenic Arabidopsis plants overexpressing H-PIF3, H-PIF3-U1 or H-PIF3-U2 with or without MG132. Lines #1 and #2 of each genotype were analyzed and PIF3 levels were detected by anti-HA antibodies. Tubulin levels were used as a loading control.

FIGS. 5a-5c show that Arabidopsis PIF3 is stabilized by UBA1/2 fusion. FIG. 5a: PIF3 protein decay is delayed by UBA1 or UBA2 fusion. Two-week old transgenic Arabidopsis plants (H-PIF3, line #1; H-PIF3-U1, line #1; and H-PIF3-U2, line #1) were incubated in liquid MS medium with MG132 (50 μM) for 12 h, washed, and then transferred to fresh MS medium with 100 μM cycloheximide (CHX) and samples were taken at different times as indicated. Proteins were analyzed by western blots as detailed in Example 1. Numbers below each lane indicate relative expression levels. Tubulin levels were used as a loading control. FIG. 5b: Time course of PIF3 protein decay after cycloheximide (CHX) treatment. Values from (a) were analyzed. FIG. 5c: Measurement of hypocotyl length of transgenic Arabidopsis seedlings expressing H-PIF3, H-PIF3-U1, and H-PIF3-U2 under R light. Two independent lines (#1 and #2) from each genotype were examined. Seedlings were grown for 4 d under R light (20 μmol m⁻² s⁻¹) on MS media without sucrose. Data were presented as average hypocotyl length±standard deviations (SD; n>40).

FIGS. 6a-6d show the characterization of 6Myc-JAZ10.1-UBA and 6Myc-JAZ10.1-UBA. FIG. 6a: Schematic diagrams of 6Myc-JAZ10.1 (M-JAZ10.1) and 6Myc-JAZ10.1-UBA (M-JAZ10.1-U). FIG. 6b: JAZ10.1 transcript levels of transgenic Arabidopsis plants (M-JAZ10.1 and M-JAZ10.1-U). Three independent lines (#1, #2 and #3) of each genotype with comparable JAZ10.1 transcript levels were used in this analysis. FIG. 6c: Western blot analysis of JAZ10.1 levels (α-Myc) in transgenic Arabidopsis plants overexpressing M-JAZ10.1 or M-JAZ10.1-U with or without MG132. Lines #1, #2 and #3 of each genotype were analyzed and JAZ10.1 levels were detected by anti-Myc antibodies. Tubulin levels were used as a loading control. FIG. 6d: Comparison of JAZ10.1 expression in Arabidopsis plants overexpressing M-JAZ10.1 and M-JAZ10.1-U. Three independent lines (#1, #2 and #3) of each genotype were analyzed. The UBA domain was derived from the DDI1 gene of Arabidopsis thalania (FIG. 8)

FIGS. 7a-7e show that Arabidopsis JAZ10.1 is stabilized by UBA1 fusion. FIG. 7a and FIG. 7c: JAZ10.1 protein decay is delayed by UBA fusion. Two-week old transgenic Arabidopsis plants (M-JAZ10.1 and M-JAZ10.1-U) were incubated in liquid MS medium with MG132 (50 μM) for 12 h, washed, and then transferred to fresh MS medium with 100 μM cycloheximide (CHX) plus 0.01 μM (FIG. 7a) or 0.1 μM (FIG. 7c) coronatine as indicated. Proteins were analyzed by western blots as detailed in Example 1. Asterisks indicate non-specific bands. Tubulin levels were used as a loading control. FIG. 7b and FIG. 7d: Time course of JAZ10.1 protein decay after cycloheximide (CHX) treatment plus 0.01 μM (FIG. 7b) or 0.1 μM (FIG. 7d) coronatine as indicated. Values from (FIG. 7a) were analyzed. FIG. 7e: Comparison of primary root length of transgenic Arabidopsis seedlings expressing M-JAZ10.1 and M-JAZ10.1-U with MeJA treatment. Three independent lines (#1, #2, and #3) of each transgenic genotype were used. Seedlings were grown for 7 d in MS media containing MeJA (5 or 20 μM). WT (col-0) and jai3-1 were used as controls. Data were presented as average root length±standard deviations (SD; n=15).

FIGS. 8a-8c show sequence comparisons of UBA domains. Amino acid sequence alignment of the UBA1 (FIG. 8a) and UBA2 (FIG. 8b) domains of RAD23a-d and UBA (FIG. 8c) of DDI1. The UBA1, UBA2 or UBA amino acid sequences of RAD23a-d or DDI1 were obtained from SMART (http://smart.embl-heidelberg.de) and analysis was performed using the MegAlign of DNAStar program. Arrows with filled and open boxes indicate MGF/Y loop (Met-Gly-Phe/Tyr) and Leu for UBA-ub interaction. FIG. 8a sequences: ScRAD23: SEQ ID NO:7; HHR23A: SEQ ID NO:8; AtRAD23a: SEQ ID NO:9; AtRAD23b: SEQ ID NO:10; AtRAD23c: SEQ ID NO:11; AtRAD23d: SEQ ID NO:12. FIG. 8b sequences: ScRAD23: SEQ ID NO:13; HHR23A: SEQ ID NO:14; AtRAD23a: SEQ ID NO:15; AtRAD23b: SEQ ID NO:16; AtRAD23c: SEQ ID NO:17; AtRAD23d: SEQ ID NO:18. FIG. 8c sequences: ScDdi1: SEQ ID NO:25; AtDDI1: SEQ ID NO:20.

FIG. 9 shows phenotypes of WT (Col), phyA-211, hfr1-201, and transgenic lines expressing M-HFR1, M-HFR1-U1, and M-HFR1-U2 under FR light (1 μmol m⁻² s⁻¹). Two independent lines (#1 and #2) of each genotype were used for the analysis. Bar represents 2 mm.

FIG. 10 shows phenotypes of WT (Col), phyB-9, pif3-3, and transgenic lines expressing H-PIF3, H-PIF3-U1, and H-PIF3-U2 under R light (20 μmol m⁻² s⁻¹). Two independent lines (#1 and #2) of each genotype were analyzed. Bar represents 5 mm.

FIG. 11 shows phenotypes of WT (Col), jar3-1, and transgenic lines expressing M-JAZ10.1 (lines #1-3) and M-JAZ10.1-U (lines #1-3) in MS media with MeJA as indicated. Bar represents 5 mm.

### DETAILED DESCRIPTION OF THE INVENTION

Unless defined otherwise, all technical and scientific terms used herein have the same meaning as is commonly understood by one of skill in the art to which the invention belongs.

The terms “polynucleotide,” “nucleotide sequence,” and “nucleic acid” are used to refer to a polymer of nucleotides (A, C, T, U, G, etc. or naturally occurring or artificial nucleotide analogues), e.g., DNA or RNA, or a representation thereof, e.g., a character string, etc., depending on the relevant context. A given polynucleotide or complementary polynucleotide can be determined from any specified nucleotide sequence.

Similarly, an “amino acid sequence” is a polymer of amino acids (a protein, polypeptide, etc.) or a character string representing an amino acid polymer, depending on context. The terms “protein,” “polypeptide,” and “peptide” are used interchangeably herein.

A polynucleotide, polypeptide or other component is “isolated” when it is partially or completely separated from components with which it is normally associated (other proteins, nucleic acids, cells, synthetic reagents, etc.). A nucleic acid or polypeptide is “recombinant” when it is artificial or engineered, or derived from an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

The term “encoding” refers to the ability of a nucleotide sequence to code for one or more amino acids. The term does not require a start or stop codon. An amino acid sequence can be encoded in any one of six different reading frames provided by a polynucleotide sequence and its complement. It will be appreciated by those skilled in the art that due to the degeneracy of the genetic code, a multitude of nucleotide sequences encoding polypeptides of the invention may be produced, some of which bear substantial identity to the nucleic acid sequences explicitly disclosed herein.

The term “nucleic acid construct” or “polynucleotide construct” means a nucleic acid molecule, either single- or double-stranded, which is isolated from a naturally occurring gene or which has been modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

The term “control sequences” is defined herein to include all components, which are necessary or advantageous for the expression of a polypeptide of the present invention. Each control sequence may be native or foreign to the nucleotide sequence encoding the polypeptide. Such control sequences include, but are not limited to, a leader sequence, polyadenylation sequence, propeptide sequence, promoter sequence, signal peptide sequence, and transcription terminator sequence. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

The term “operably linked” is defined herein as a configuration in which a control sequence is appropriately placed at a position relative to the coding sequence of the DNA sequence such that the control sequence directs the expression of a polypeptide.

When used herein the term “coding sequence” is intended to cover a nucleotide sequence, which directly specifies the amino acid sequence of its protein product. The boundaries of the coding sequence are generally determined by an open reading frame, which usually begins with the ATG start codon. The coding sequence typically includes a DNA, cDNA, and/or recombinant nucleotide sequence.

In the present context, the term “expression” includes any step involved in the production of the polypeptide including, but not limited to, transcription, post-transcriptional modification, translation, post-translational modification, and secretion. In the present context, the term “expression vector” covers a DNA molecule, linear or circular, that comprises a segment encoding a polypeptide of the invention, and which is operably linked to additional segments that provide for its transcription.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

The term “heterologous” as used herein describes a relationship between two or more elements which indicates that the elements are not normally found in proximity to one another in nature. Thus, for example, a polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). An example of a heterologous polypeptide is a polypeptide expressed from a recombinant polynucleotide in a transgenic organism. Heterologous polynucleotides and polypeptides are forms of recombinant molecules.

The term “transfecting” as used herein refers to the deliberate introduction to a nucleic acid into a cell. Transfection includes any method known to the skilled artisan for introducing a nucleic acid into a cell, including, but not limited to, Agrobacterium infection, ballistics, electroporation, microinjection and the like.

The term “more stable” as used herein means that the fusion protein comprising the protein of interest and a UBA domain described herein is at least 3 times, preferably at least 4 times, and more preferably at least 5 times more stable as measured by half-life of the fusion protein in a transgenic plant cell or transgenic plant than the corresponding protein of interest under similar conditions. This greater stability enables the fusion protein to impart an enhanced phenotype to the transgenic plant cell or transgenic plant compared to the protein of interest.

The final expression level of a transgene-derived protein in transgenic plants depends on transcriptional and post-transcriptional processes. The present invention focuses on compositions and methods to improve protein stability without compromising biological function. As shown herein, the 4 isoforms of the Arabidopsis RAD23 protein family are relatively stable. As also shown herein, the UBA2 domain derived from Arabidopsis RAD23a is used as a portable stabilizing signal to prolong half-life of two unstable transcription factors (TFs), HFR1 and PIF3. Increased stability of the TF-UBA2 fusion protein results in an enhanced phenotype in transgenic plants as compared to expression of the TF alone. Similar results are shown herein for the Arabidopsis RAD23a UBA1 domain. In addition to UBA1/2 of RAD23, a UBA from Arabidopsis DDI1 protein also could increase the stability of unstable protein, JAZ10.1 by C-terminal fusion. Taken together, our results demonstrate that UBA1/2 fusions can be used for increasing stability of unstable proteins in transgenic plants developed for plant biotechnology, such as crop improvement and the production of foreign protein in plants and plant tissue cultures.

Thus, the present invention provides compositions and methods for enhancing protein stability in transgenic plants. The compositions are nucleic acid constructs which encode fusion proteins, fusion proteins, transgenic plant cells and transgenic plants. A fusion protein in accordance with the present invention comprises a protein of interest and a UBA domain of a suitable protein. The methods use the nucleic acid constructs to produce fusion proteins in transgenic plant cells or transgenic plants. The fusion proteins have greater stability than the protein of interest and have the same function as the proteins of interest.

In a first aspect, the present invention provides a nucleic acid construct comprising a plant operable promoter as described herein operably linked to a nucleic acid encoding a fusion protein. The nucleic acid construct may optionally include other regulatory sequences as described herein. In one embodiment, the nucleic acid encoding a fusion protein comprises a first nucleic acid segment and a second nucleic acid segment. In another embodiment, the first nucleic acid segment is a DNA of interest which encodes a protein of interest as described herein. In accordance with a goal of the present invention, i.e., the enhancement of protein stability, the protein of interest is preferably a protein that is unstable when expressed in a transgenic plant cell or a transgenic plant. Further in accordance with the present invention, the fusion protein has the same function as the corresponding protein of interest.

The second nucleic acid segment has a nucleotide sequence that encodes a UBA domain described herein. In one embodiment, the UBA domain is a UBA domain of a suitable protein described herein. Such nucleotide sequences can be readily prepared by the skilled artisan on the basis of the amino acid sequence of a UBA1 domain described herein and the degeneracy of the genetic code. In one embodiment, the first nucleic acid segment is operatively linked to the second nucleic acid segment such that upon expression a fusion protein is produced having the protein of interest as the N-terminal segment of the fusion protein and the UBA domain as the C-terminal segment. In this embodiment, the UBA domain is fused to the C-terminus of the protein of interest.

In one embodiment, the suitable protein is an Arabidopsis RAD23a protein (GenBank Accession No. NP_{—}173070). In another embodiment, the suitable protein is an Arabidopsis RAD23b protein (GenBank Accession No. NP_{—}850982). In an additional embodiment, the suitable protein is an Arabidopsis RAD23c protein (GenBank Accession No. NP_{—}186903). In a further embodiment, the suitable protein is an Arabidopsis RAD23d protein (GenBank Accession No. NP_{—}198663). In one embodiment, the UBA domain is a UBA1 domain of one of the RAD23 proteins. In another embodiment, the UBA domain is a UBA2 domain of one of the RAD23 proteins. In one embodiment, the suitable protein is a yeast RAD23 protein (GenBank Accession No. AAB28441). In another embodiment, the suitable protein is a yeast Ddi1 protein (GenBank Accession No. NP_{—}011070). In an additional embodiment, the suitable protein is an Arabidopsis UBL1 protein (GenBank Accession No. NP_{—}197113). In a further embodiment, the suitable protein is an Arabidopsis DDI1 protein (GenBank Accession No. ABG25069). In another embodiment, the suitable protein is an Arabidopsis DSK2a protein (GenBank Accession No. AAM10012). In an additional embodiment, the suitable protein is an Arabidopsis DSK2b protein (GenBank Accession No. AAN13037). In a further embodiment, the suitable protein is an Arabidopsis NUB1 protein (GenBank Accession No. AAY34175). In one embodiment, the UBA domain is a UBA1 domain of the NUB1 protein. In another embodiment, the UBA domain is a UBA2 domain of the NUB1 protein.

In a first embodiment, the UBA domain is a UBA1 domain of an Arabidopsis RAD23 protein. In accordance with this embodiment, the UBA domain has an amino acid sequence selected from the group of peptides set forth in SEQ ID NO:1, SEQ ID NO:9, SEQ ID NO:10, SEQ ID NO:11 and SEQ ID NO:12. As used herein these sequences of a UBA1 domain of an Arapidopsis RAD23 protein are referred to as native sequences or domains.

In a second embodiment, the UBA domain is a UBA2 domain of an Arabidopsis RAD23 protein. In accordance with this embodiment, the UBA domain has an amino acid sequence selected from the group of peptides set forth in SEQ ID NO:2, SEQ ID NO:15, SEQ ID NO:16, SEQ ID NO:17 and SEQ ID NO:18. As used herein these sequences of a UBA2 domain of an Arapidopsis RAD23 protein are referred to as native sequences or domains.

In a third embodiment, the UBA domain is a UBA2 domain of a yeast RAD23 protein. In accordance with this embodiment, the UBA domain has the amino acid sequence set forth in SEQ ID NO:13. As used herein this sequence of a UBA2 domain of a yeast RAD23 protein is referred to as a native sequence or domain.

In a fourth embodiment, the UBA domain is a UBA domain of an Arabidopsis UBL1 protein. In accordance with this embodiment, the UBA domain has the amino acid sequence set forth in SEQ ID NO:19. As used herein this sequence of a UBA domain of a Arabidopsis UBL1 protein is referred to as a native sequence or domain.

In a fifth embodiment, the UBA domain is a UBA domain of an Arabidopsis DDI1 protein. In accordance with this embodiment, the UBA domain has the amino acid sequence set forth in SEQ ID NO:20. As used herein this sequence of a UBA domain of a Arabidopsis DDI1 protein is referred to as a native sequence or domain.

In a sixth embodiment, the UBA domain is a UBA domain of a yeast Ddi1 protein. In accordance with this embodiment, the UBA domain has the amino acid sequence set forth in SEQ ID NO:25. As used herein this sequence of a UBA domain of a yeast Ddi1 protein is referred to as a native sequence or domain.

In a seventh embodiment, the UBA domain is a UBA domain of an Arabidopsis DSK2a protein. In accordance with this embodiment, the UBA domain has the amino acid sequence set forth in SEQ ID NO:21. As used herein this sequence of a UBA domain of a Arabidopsis DSK2a protein is referred to as a native sequence or domain.

In an eighth embodiment, the UBA domain is a UBA domain of an Arabidopsis DSK2b protein. In accordance with this embodiment, the UBA domain has the amino acid sequence set forth in SEQ ID NO:22. As used herein this sequence of a UBA domain of a Arabidopsis DSK2b protein is referred to as a native sequence or domain.

In a ninth embodiment, the UBA domain is a UBA1 domain of an Arabidopsis NUB1 protein. In accordance with this embodiment, the UBA domain has the amino acid sequence set forth in SEQ ID NO:23. As used herein this sequence of a UBA domain of a Arabidopsis NUB1 protein is referred to as a native sequence or domain.

In an tenth embodiment, the UBA domain is a UBA2 domain of an Arabidopsis NUB1 protein. In accordance with this embodiment, the UBA domain has the amino acid sequence set forth in SEQ ID NO:24. As used herein this sequence of a UBA domain of a Arabidopsis NUB 1 protein is referred to as a native sequence or domain.

In an eleventh embodiment, the UBA domain is a UBA1 domain of a Populus NUB 1 protein. In accordance with this embodiment, the UBA domain has the amino acid sequence set forth in SEQ ID NO:26. As used herein this sequence of a UBA domain of a Populus NUB1 protein is referred to as a native sequence or domain.

In a twelfth embodiment, the UBA domain is a UBA2 domain of a Populus NUB1 protein. In accordance with this embodiment, the UBA domain has the amino acid sequence set forth in SEQ ID NO:27. As used herein this sequence of a UBA domain of a Populus NUB1 protein is referred to as a native sequence or domain.

In a thirteenth embodiment, the UBA domain is a UBA1 domain of a Ricinis NUB1 protein. In accordance with this embodiment, the UBA domain has the amino acid sequence set forth in SEQ ID NO:28. As used herein this sequence of a UBA domain of a Ricinis NUB1 protein is referred to as a native sequence or domain.

In a fourteenth embodiment, the UBA domain is a UBA2 domain of a Ricinis NUB 1 protein. In accordance with this embodiment, the UBA domain has the amino acid sequence set forth in SEQ ID NO:29. As used herein this sequence of a UBA domain of a Ricinis NUB1 protein is referred to as a native sequence or domain.

In a fifteenth embodiment, the UBA domain is a UBA2 domain of a Vitis NUB1 protein. In accordance with this embodiment, the UBA domain has the amino acid sequence set forth in SEQ ID NO:30. As used herein this sequence of a UBA domain of a Vitis NUB1 protein is referred to as a native sequence or domain.

In a sixteenth embodiment, the UBA domain is a UBA2 domain of a Oryza NUB1 protein. In accordance with this embodiment, the UBA domain has the amino acid sequence set forth in SEQ ID NO:31. As used herein this sequence of a UBA domain of a Oryza NUB1 protein is referred to as a native sequence or domain.

In seventeenth embodiment, the UBA domain is a UBA2 domain of a Sorghum NUB 1 protein. In accordance with this embodiment, the UBA domain has the amino acid sequence set forth in SEQ ID NO:32. As used herein this sequence of a UBA domain of a Sorghum NUB 1 protein is referred to as a native sequence or domain.

In another embodiment, the UBA domain has an amino acid sequence that is a conservatively modified variation. A “conservatively modified variation” of a UBA domain is an amino acid sequence having individual substitutions, deletions or additions which alter, add or delete a single amino acid or a small percentage of amino acids (typically less than 5%, more typically less than 4%, 3%, 2% or 1%, or less) in the amino acid sequence. A “conservatively modified variation” of a UBA domain is also an amino acid sequence having greater than 95%, 96%, 97%, 98% or 99% sequence identity with a native UBA domain. A “conservatively modified variation” of a UBA domain is also an amino acid sequence having one or two amino acid changes with respect to a native UBA domain, wherein the change is selected from the group consisting of substitutions, deletions and additions. The alterations result in the deletion of an amino acid, addition of an amino acid, or substitution of an amino acid with a “conservative amino acid substitution.” A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid, asparagine, glutamine), uncharged polar side chains (e.g., glycine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). A “conservatively substituted variation” of a UBA domain of the present invention includes substitutions of a small percentage, typically 5% or less of the amino acids of the UBA domain sequence, with a conservative amino acid substitution. As used herein, each of the above conservatively modified variation or conservatively substituted variation is termed a “conservative variant”. In accordance with the present invention, a conserved variant of a UBA domain has the same function as a native UBA domain.

The term “UBA domain” as used herein is intended to encompass a native UBA domain and a conservative variant of a native UBA domain, unless the context dictates otherwise. The term “UBA domain” also encompasses a chimeric domain comprising two or more fragments of native UBA domains, in which the chimeric domain is capable of stabilizing a protein of interest when fused to the carboxy-terminal end of said protein of interest.

In a second aspect, the present invention provides a fusion protein. In one embodiment, the fusion protein comprises a first protein segment and a second protein segment. In another embodiment, the first protein segment is a protein of interest. In an additional embodiment, the second protein segment is a UBA domain as described herein, which, as described above, includes a native UBA domain or a conservative variant of a UBA domain. In one embodiment, the UBA domain is fused to the C-terminus of the protein of interest. In accordance with the present invention, the fusion protein has the same function as the corresponding protein of interest.

In another embodiment, the two protein domains, e.g., the first and second protein segments of the fusion protein, are present as inteins that are encoded on different expression units, and the fusion protein is formed by in vivo protein splicing of the two inteins. The two expression units encoding the inteins may have different promoters, e.g., the protein of interest may be constitutively expressed and the UBA domain may be expressed in a tissue-specific or inducible fashion. See, for example Yang et al. (2003); U.S. Pat. No. 7,906,704.

In a third aspect, the present invention provides a transgenic plant cell comprising the nucleic acid construct. In one embodiment, the nucleic acid construct is stably integrated into the genome of the transgenic plant cell. In one embodiment, the fusion protein is expressed in the transgenic plant cell. In another embodiment, the fusion protein is more stable in the transgenic plant cell than the corresponding protein of interest. In an additional embodiment, the fusion protein has the same function in the transgenic plant cell as the corresponding protein of interest. The transgenic plant cell is prepared by transfecting a plant cell with a nucleic acid construct using methods well known in the art including, but not limited to, those described herein. Plant cells of a wide variety of plant species can be transfected with a nucleic acid construct of the present invention. A plant cell containing the nucleic acid construct is selected in accordance with conventional techniques including, but not limited to, those described herein. The plant cell is grown under conditions suitable for the expression of the nucleic acid in the transfected plant cell using growth conditions well known in the art.

The present invention may be used for transfecting plant cells of a wide variety of plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago saliva), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setara italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats (Avena sativa), barley (Hordeum vulgare), switchgrass (Panicum virgatum), vegetables, ornamentals, and conifers. See U.S. Pat. No. 7,763,773 for a list of additional plant species that can be used in accordance with the present invention.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pukhernima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesil); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

In a fourth aspect, the present invention provides a transgenic plant comprising the nucleic acid construct. In one embodiment, the nucleic acid construct is stably integrated into the genome of the transgenic plant. In one embodiment, the fusion protein is expressed in the transgenic plant. In another embodiment, the fusion protein is more stable in the transgenic plant than the corresponding protein of interest. In an additional embodiment, the fusion protein has the same function in the transgenic plant as the corresponding protein of interest. Transgenic plants are regenerated from transgenic plant cells described herein using conventional techniques well known to the skilled artisan using various pathways, including somatic embryogenesis and organogenesis. Transformed plant cells which are derived by plant transformation techniques, including those discussed above, can be cultured to regenerate a whole plant which possesses the transformed genotype, and thus the desired phenotype. Such regeneration techniques generally rely on manipulation of certain phytohormones in a tissue culture growth medium, typically relying on a marker which has been introduced together with the desired nucleotide sequences. See, for example, U.S. Pat. No. 7,763,773, U.S. Patent Application Publication No. 2010/0199371 and International Published Application No. WO 2008/094127 and references cited therein. The transgenic plant is grown under conditions suitable for the expression of the nucleic acid in the transfected plant using growth conditions well known in the art.

In a fifth aspect, the present invention provides a method of enhancing the stability of a protein of interest in a transgenic plant cell or in a transgenic plant. In one embodiment, the method comprises transfecting a plant cell with the nucleic acid construct to produce a transgenic plant cell as described herein. The method further comprises expressing the fusion protein in the transgenic plant cell as described herein. The expressed fusion protein is more stable in the transgenic plant cell than the corresponding protein of interest and has the same function in the transgenic plant cell as the corresponding protein of interest. The method may optionally include preparing a nucleic acid construct encoding a fusion protein as described herein. In another embodiment, the method comprises regenerating a transgenic plant from the transgenic plant cell as described herein. In this embodiment, the fusion protein is expressed in the transgenic plant as described herein. The expressed fusion protein is more stable in the transgenic plant than the corresponding protein of interest and has the same function in the transgenic plant as the corresponding protein of interest.

The DNA that encodes the protein of interest and that is inserted (the DNA of interest) into plants in accordance with the present invention is not critical to the transformation process. Generally the DNA that is introduced into a plant is part of a construct as described herein. The construct typically includes regulatory regions operatively linked to the 5′ side of the DNA of interest and/or to the 3′ side of the DNA of interest. A cassette containing all of these elements is also referred to herein as an expression cassette. The expression cassettes may additionally contain 5′ leader sequences in the expression cassette construct. The regulatory regions (i.e., promoters, transcriptional regulatory regions, and translational termination regions) and/or the polynucleotide encoding a signal anchor may be native/analogous to the host cell or to each other. Alternatively, the regulatory regions and/or the polynucleotide encoding a signal anchor may be heterologous to the host cell or to each other. See, U.S. Pat. Nos. 7,205,453 and 7,763,773, and U.S. Patent Application Publication Nos. 2006/0218670, 2006/0248616 and 20090100536, and the references cited therein. The expression cassettes may additionally contain 5′ leader sequences in the expression cassette construct. Such leader sequences can act to enhance translation. Translation leaders are known in the art and include those described in International Publication No. WO 2008/094127 and the references cited therein.

The DNA of interest that is under control of a promoter may be any DNA as defined herein and may be used to alter any characteristic or trait of a plant species into which it is introduced. The DNA of interest may encode any protein (protein of interest) that is desired to be expressed in a transgenic plant cell or a transgenic plant. In one embodiment, the protein of interest is a protein that is to be expressed in a plant cell or a plant. The protein may be a regulatory protein, such as a transcription factor and the like, a binding or interacting protein, or a protein that alters a phenotypic trait of a transgenic plant cell or a transgenic plant. In one embodiment, the DNA of interest is introduced into a plant in order to enhance a trait of the plant. In another embodiment, an enhanced agronomic trait may be characterized by enhanced plant morphology, physiology, growth and development, yield, nutritional enhancement, disease or pest resistance, or environmental or chemical tolerance. In some aspects, the enhanced trait is selected from group of enhanced traits consisting of enhanced water use efficiency, enhanced temperature tolerance, increased yield, enhanced nitrogen use efficiency, enhanced seed protein enhanced seed oil and enhanced biomass. Increase yield may include increased yield under non-stress conditions and increased yield under environmental stress conditions. Stress conditions may include, for example, drought, shade, fungal disease, viral disease, bacterial disease, insect infestation, nematode infestation, extreme temperature exposure (cold or hot), osmotic stress, reduced nitrogen nutrient availability, reduced phosphorus nutrient availability and high plant density. In some embodiments, the DNA of interest may be used to modify metabolic pathways, such as fatty acid biosynthesis or lipid biosynthesis pathways in seeds, or to modify resistance to pathogens in plants.

Generally, the expression cassette may additionally comprise a selectable marker gene for the selection of transformed cells. Selectable marker genes are utilized for the selection of transformed cells or tissues. Usually, the plant selectable marker gene will encode antibiotic resistance, with suitable genes including at least one set of genes coding for resistance to the antibiotic spectinomycin, the streptomycin phosphotransferase (spt) gene coding for streptomycin resistance, the neomycin phosphotransferase (nptII) gene encoding kanamycin or geneticin resistance, the hygromycin phosphotransferase (hpt or aphiv) gene encoding resistance to hygromycin, acetolactate synthase (als) genes. Alternatively, the plant selectable marker gene will encode herbicide resistance such as resistance to the sulfonylurea-type herbicides, glufosinate, glyphosate, ammonium, bromoxynil, imidazolinones, and 2,4-dichlorophenoxyacetate (2,4-D), including genes coding for resistance to herbicides which act to inhibit the action of glutamine synthase such as phosphinothricin or basta (e.g., the bar gene). See generally, International Publication No. WO 02/36782, U.S. Pat. Nos. 7,205,453 and 7,763,773, and U.S. Patent Application Publication Nos. 2006/0218670, 2006/0248616, 2007/0143880 and 20090100536, and the references cited therein. See also, Jefferson et al. (1991); De Wet et al. (1987); Goff et al. (1990); Kain et al. (1995) and Chiu et al. (1996). This list of selectable marker genes is not meant to be limiting. Any selectable marker gene can be used. The selectable marker gene is also under control of a promoter operable in the plant species to be transformed. Such promoters include those described in International Publication No. WO 2008/094127 and the references cited therein. See also, U.S. Patent Application Publication Nos. 2008/0313773 and 2010/0199371 for an exemplification of additional markers that can be used in accordance with the present invention.

A number of promoters can be used in the practice of the invention. The promoters can be selected based on the desired outcome. That is, the nucleic acids can be combined with constitutive, tissue-preferred, or other promoters for expression in the host cell of interest. Such constitutive promoters include, for example, the core promoter of the Rsyn7 (WO 99/48338 and U.S. Pat. No. 6,072,050); the core CaMV35S promoter (Odell et al., 1985); rice actin (McElroy et al., 1990); ubiquitin (Christensen and Quail, 1989; Christensen et al., 1992); pEMU (Last et al., 1991); MAS (Velten et al., 1984); ALS promoter (U.S. Pat. No. 5,659,026), and the like. Other constitutive promoters include, for example, those disclosed in U.S. Pat. Nos. 5,608,149; 5,608,144; 5,604,121; 5,569,597; 5,466,785; 5,399,680; 5,268,463; and 5,608,142.

Other promoters include inducible promoters. Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners Pathogen-inducible promoters include those from pathogenesis-related proteins (PR proteins), which are induced following infection by a pathogen; e.g., PR proteins, SAR proteins, beta-1,3-glucanase, chitinase, etc. Other promoters include those that are expressed locally at or near the site of pathogen infection. In further embodiments, the promoter may be a wound-inducible promoter. In other embodiments, chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. In addition, tissue-preferred promoters can be utilized to target enhanced expression of a polynucleotide of interest within a particular plant tissue. Each of these promoters is described in U.S. Pat. Nos. 6,506,962, 6,575,814, 6,972,349 and 7,301,069 and in U.S. Patent Application Publication Nos. 2007/0061917 and 2007/0143880. See also, U.S. Patent Application Publication Nos. 2008/0313773 and 2010/0199371 for an exemplification of additional promoters that can be used in accordance with the present invention.

Promoters for use in the current invention may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (U.S. Patent Application Publication No. 2006/0156439), the maize ROOTMET2 promoter (International Publication No. WO 05/063998), the CR1BIO promoter (International Publication No. WO 06/055487), the CRWAQ81 promoter (International Publication No. WO 05/035770) and the maize ZRP2.47 promoter (NCBI accession number: U38790; GI No. 1063664).

Where appropriate, the DNA of interest may be optimized for increased expression in the transformed plant. That is, the coding sequences can be synthesized using plant-preferred codons for improved expression. Methods are available in the art for synthesizing plant-preferred genes. See, for example, U.S. Pat. Nos. 5,380,831, 5,436,391, and 7,205,453 and U.S. Patent Application Publication Nos. 2006/0218670 and 2006/0248616.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g. transitions and transversions may be involved.

Once a nucleic acid has been cloned into an expression vector, it may be introduced into a plant cell using conventional transformation (or transfection) procedures. The term “plant cell” is intended to encompass any cell derived from a plant including undifferentiated tissues such as callus and suspension cultures, as well as plant seeds, pollen or plant embryos. Plant tissues suitable for transformation include leaf tissues, root tissues, meristems, protoplasts, hypocotyls, cotyledons, scutellum, shoot apex, root, immature embryo, pollen, and anther. “Transformation” means the directed modification of the genome of a cell by the external application of recombinant DNA from another cell of different genotype, leading to its uptake and integration into the subject cell's genome. In this manner, genetically modified plants, plant cells, plant tissue, seed, and the like can be obtained.

DNA constructs in accordance with the present invention can be used to transform any plant. The constructs may be introduced into the genome of the desired plant host by a variety of conventional techniques. Techniques for transforming a wide variety of higher plant species are well known and described in the technical and scientific literature. Transformation protocols may vary depending on the type of plant or plant cell, i.e., monocot or dicot, targeted for transformation, as is well known to the skilled artisan. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Thus, any method, which provides for effective transformation/transfection may be employed. See, for example, U.S. Pat. Nos. 7,241,937, 7,273,966 and 7,291,765 and U.S. Patent Application Publication Nos. 2007/0231905 and 2008/0010704 and references cited therein. See also, International Published Application Nos. WO 2005/103271 and WO 2008/094127 and references cited therein. See also, U.S. Patent Application Publication Nos. 2008/0313773 and 2010/0199371 for an exemplification of transformation protocols for a variety of plant species that can be used in accordance with the present invention.

Transformed plant cells which are derived by any of the above transformation techniques can be cultured to regenerate a whole plant which possesses the transformed genotype and thus the desired phenotype, e.g., a transgenic plant. A “transgenic plant” is a plant into which foreign DNA has been introduced. A “transgenic plant” encompasses all descendants, hybrids, and crosses thereof, whether reproduced sexually or asexually, and which continue to harbor the foreign DNA. Regeneration techniques rely on manipulation of certain phytohormones in a tissue culture growth medium, typically relying on a biocide and/or herbicide marker which has been introduced together with the desired nucleotide sequences. See for example, International Published Application No. WO 2008/094127 and references cited therein and U.S. Patent Application Publication No. 2010/0199371.

The foregoing methods for transformation are typically used for producing a transgenic variety in which the expression cassette is stably incorporated. After the expression cassette is stably incorporated in transgenic plants, it can be transferred to other plants by sexual crossing. In one embodiment, the transgenic variety could then be crossed, with another (non-transformed or transformed) variety, in order to produce a new transgenic variety. Alternatively, a genetic trait which has been engineered into a particular cotton line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties which do not contain that gene. As used herein, “crossing” can refer to a simple X by Y cross, or the process of backcrossing, depending on the context. Any of a number of standard breeding techniques can be used, depending upon the species to be crossed.

Once transgenic plants of this type are produced, the plants themselves can be cultivated in accordance with conventional procedures. Transgenic seeds can, of course, be recovered from the transgenic plants. These seeds can then be planted in the soil and cultivated using conventional procedures to produce transgenic plants. The cultivated transgenic plants will express the DNA of interest as described herein.

In another aspect, the present invention provides plant cell bioreactors for the production of a protein of interest. The enhanced stability of the fusion proteins of the present invention enable greater recovery of the protein of interest in the plant cell bioreactors. In this aspect, the nucleic acid construct may be modified to include a third nucleic acid segment located between the first and second nucleic acid segments. The third nucleic acid segment encodes a cleavage sequence, i.e., an amino acid sequence that is recognized and cleaved by a suitable enzyme or by suitable chemical means. The cleavage sequence is selected such that it is not present in the protein of interest. Cleavage sequences are well known in the art, and include, but are not limited to those described by LaVallie et al. (2001). Expression of the nucleic acid construct containing the three nucleic acid segments will produce a fusion protein comprising the protein of interest as the N-terminal segment followed by the cleavage sequence followed by the UBA domain.

Transgenic plant cells are prepared as described above and are then used in a plant cell bioreactor. For a description of plant cell bioreactors, see U.S. Patent Application Publication Nos. 2006/0248616 and 2006/0218670. For a discussion of plant cell bioreactors and production of proteins in such bioreactors, see U.S. Patent Application Publication Nos. 2010/0299787, 2006/0248616 and 2006/0218670 and James and Lee (2001). The plant cells in the plant cell bioreactor are cultured under conditions suitable to express the fusion protein. The fusion proteins produced in a plant bioreactor can be isolated and/or purified in accordance with techniques well known in the art. Various techniques suitable for use in protein purification are well known to the skilled artisan. These include, for example, precipitation with ammonium sulphate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques known to the skilled artisan. After isolation and/or purification, the fusion protein containing a cleavage sight can be cleaved and the protein of interest recovered. Alternatively, such a fusion protein can be cleaved before isolation and/or purification.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Russell, 1984, Molecular biology of plants: a laboratory course manual (Cold Spring Harbor Laboratory Press, Cold Spring Harbor, N.Y.); Anand, Techniques for the Analysis of Complex Genomes, (Academic Press, New York, 1992); Guthrie and Fink, Guide to Yeast Genetics and Molecular Biology (Academic Press, New York, 1991); Harlow and Lane, 1988, Antibodies, (Cold Spring Harbor Laboratory Press, Cold Spring Harbor, N.Y.); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Fire et al., RNA Interference Technology: From Basic Science to Drug Development, Cambridge University Press, Cambridge, 2005; Schepers, RNA Interference in Practice, Wiley-VCH, 2005; Engelke, RNA Interference (RNAi): The Nuts & Bolts of siRNA Technology, DNA Press, 2003; Gott, RNA Interference, Editing, and Modification: Methods and Protocols (Methods in Molecular Biology), Human Press, Totowa, N.J., 2004; Sohail, Gene Silencing by RNA Interference: Technology and Application, CRC, 2004.

### EXAMPLES

The present invention is described by reference to the following Examples, which are offered by way of illustration and are not intended to limit the invention in any manner. Standard techniques well known in the art or the techniques specifically described below were utilized.

### Example 1

### Materials and Methods

Vector Construction:

DNA fragments encoding UBA1 and UBA2 domains were amplified by PCR from Arabidopsis RAD23a (AT1G16190; GenBank Accession No. NM_{—}101486) cDNA. UBA1 contains amino acids 143-186 (GSSIEQMVQQIMEMGGGSWDK ETVTRALRAAYNNPERAVDYLYS; SEQ ID NO:1) of RAD23a and UBA2 contains amino acids 323-361 (EEQESIERLEAMGFDRAIVIEAFLSCDRNEELAANYLLE; SEQ ID NO:2) of RAD23a. Both UBA1 and UBA2 DNA fragments were fused to 3′ end of full length HFR1 or PIF3 cDNA to generate HFR1-UBA1, HFR1-UBA2, PIF3-UBA1, and PIF3-UBA2. All cDNA or DNA fragments were cloned into pBA-6Myc to generate 6Myc-HFR1, 6Myc-HFR1-UBA1, and 6Myc-HFR1-UBA2 or pBA-3HA to generate 3HA-PIF3, 3HA-PIF3-UBA1, and 3HA-PIF3-UBA2. All constructs were transcribed from a CaMV 35S promoter and were verified by sequencing.

DNA fragment encoding UBA domain from Arabidopsis DDI1 (AT3G13235) were amplified by PCR. UBA of DDI1 contains amino acid 375-411 (FEAKIAKLVELFSRDSVIQA LKLFEGNEEQAAGFLFG; SEQ ID NO:20) of DDI1.

PCR amplified UBA was fused to 3′ end of full length JAZ10.1 (AT5G13220) cDNA to generate JAZ10.1-UBA. Both JAZ10.1 and JAZ10.1-UBA fragments were cloned into pBA-6Myc to generate 6Myc-JAZ10.1 and 6Myc-JAZ10.1-UBA, respectively.

Protein Extraction and Western Blotting:

Approximately 100 mg of whole Arabidopsis seedlings was frozen in liquid-N₂ and ground to a fine powder using a mortar and a pestle. The powder was re-suspended by homogenization at 4° C. in a buffer (50 mM Tris-HCl, pH 7.5; 100 mM NaCl; 0.2% Triton X-100; 1 mM DTT; 2 mM PMSF) containing proteinase inhibitor cocktail (Roche). After homogenization, the mixture was clarified by centrifugation and protein concentration was determined using the protein assay (Bio-Rad). Protein extracts (10 μg) were separated on 8% SDS-polyacrylamide gels and transferred to a polyvinylidene difluoride (PVDF) membranes (Immobilon-P, Millipore) using an electro transfer apparatus (BioRad). The membranes were incubated with anti-tubulin (Sigma) or anti-HA or anti-Myc (Santa Cruz Biotechnology) primary antibodies and peroxidase-conjugated secondary antibodies (Amersham Biosciences) before visualization of immunoreactive proteins using ECL kits (Amersham Biosciences). Tubulin levels were used as loading controls.

RNA Extraction and Quantitative RT-qPCR:

Total RNA was isolated from Arabidopsis seedlings using Qiagene RNeasy Plant Mini Kits (Qiagene). After quantification of RNA, reverse transcription was performed using an oligo(dT) and SuperScript III RT kit (Invitrogen). The cDNA was quantified using a SYBR premix Ex Taq (TaKaRa) with gene specific primers in Bio-Rad CFX96 real time system. ACTIN2 was used as an internal control. The primers were as following: 5′-gaaacgacgtcggatcactt-3′ (SEQ ID NO:3) and 5′-ttcatcagggacaacaacca-3′ (SEQ ID NO:4) for HFR1 amplification and 5′-agatttcaggctcaccaaa-3′ (SEQ ID NO:5) and 5% gcttgtg gtggaggaatgtt-3′ (SEQ ID NO:6) for PIF3 amplification, and 5′-tcggtaattatccgaccac-3′ (SEQ ID NO:33) and 5′-gccgatgtcggatagtaagg-3′ (SEQ ID NO:34) for JAZ10.1 amplification.

MG132 and Cycloheximide Treatments:

Transgenic Arabidopsis seedlings grown on MS media for two weeks (16 h L/8 h D) were treated with MG132 (50 μM) for 12 h. Treated seedlings were harvested for Western blot analyses. For protein decay experiment of HFR1 or PIF3-related proteins, seedlings treated as above were washed 3 times with MS medium and transferred to fresh MS liquid medium containing 100 μM cycloheximide (Sigma) to block new protein synthesis, kept in the dark for 30 min and then exposed to white light (200 μmol m⁻² s⁻¹).

For protein decay experiment of JAZ10.1-related proteins, seedlings treated with or without MG132 (50 μM) for 12 h were washed as above and transferred to fresh MS liquid medium containing 100 μM cycloheximide (Sigma), kept for 30 min and then treated with coronatine (Sigma) as described.

Proteins were extracted at the indicated times and analyzed by Western blotting using anti-HA or anti-Myc (Santa Cruz Biotechnology) antibodies. Protein expression levels of 6Myc-HFR1 or 3HA-PIF3 and tubulin were measured using the program of Image Gauge V3.12 (Fuji) and the values were normalized to 0 time in all panels.

Light Treatments:

We used WT (Col-0), phyA-211, phyB-9, hfr1-201, pf3-3 as controls for light treatment. All mutants were in the Col-0 background. For phenotypic analysis of transgenic seedlings, surface-sterilized WT (Col-0) and mutant seedlings on MS media that was not supplemented with sucrose were kept for 4 d at 4° C. in darkness, exposed to 1 h white light and then transferred to continuous FR (1 μmol m⁻² s⁻¹) or R (20 μmol m⁻² s⁻¹) for 4 d at 21° C. Hypocotyl lengths more than 40 seedlings of each line were measured and analyzed using Image J software (http://rsb.info.nih.gov/ij/).

MeJA Treatment for Root Growth Inhibition:

WT (Col-0) and jar3-1 (Chini et al., 2007) were used as controls for JA-sensitive and JA-insensitive phenotypes in root growth inhibition assay under methyl jasmonate (MeJA) treatment, respectively. Surface-sterized WT (Col-0), jar3-1 and JAZ10.1-related seedlings on MS medium that was supplemented with 50 μM MeJA were kept for 4d at 4° C. in darkness and then incubated normal growth condition. Plates were oriented in a vertical position for 7 to 10 days to measure root length. Root lengths of more than 15 seedlings of each line were measured and analyzed using Image J software (http://rsb.info.nih.gov/ij/).

### Example 2

### Arabidopsis RAD23 Proteins are Relatively Stable

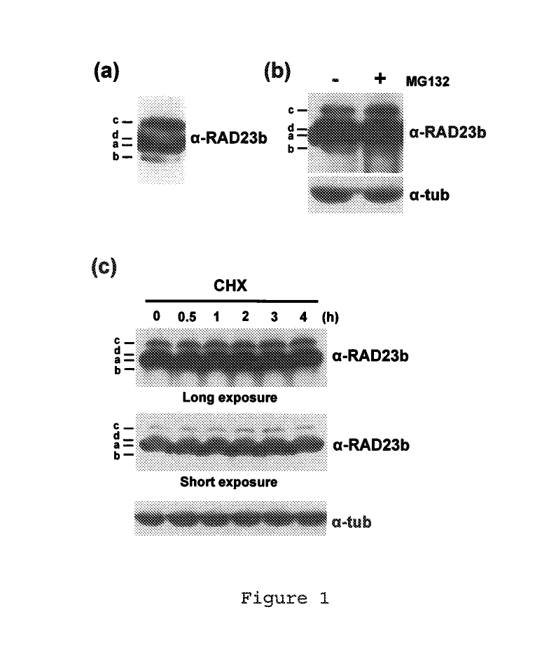

The Arabidopsis genome encodes 4 RAD23 proteins (RAD23a, b, c and d) which are highly conserved, particularly in their UBA1 and UBA2 motifs (Farmer et al., 2010). In confirmation of previous results (Farmer et al., 2010) rabbit antibody raised against Arabidopsis RAD23b was able to recognize all the 4 RAD23 isoforms (a-d) (FIG. 1a) because of the high amino acid sequence homology among them. FIG. 1b shows that the Arabidopsis RAD23 proteins were relatively stable as the expression level was not noticeably increased by the addition of MG132, which blocks protein degradation. This conclusion was reinforced by an experiment in which protein decay was directly monitored after arrest of protein synthesis by cycloheximide. Under this condition no significant change in RAD23 levels was detected within 4 hrs (FIG. 1c). Our results on the relatively stability of Arabidopsis RAD23 proteins is consistent with previous reports that the RAD23 protein of yeast and mammalian cell is very stable (Heessen et al., 2003; Heessen et al., 2005).

### Example 3

### Increasing the Stability of HFR1

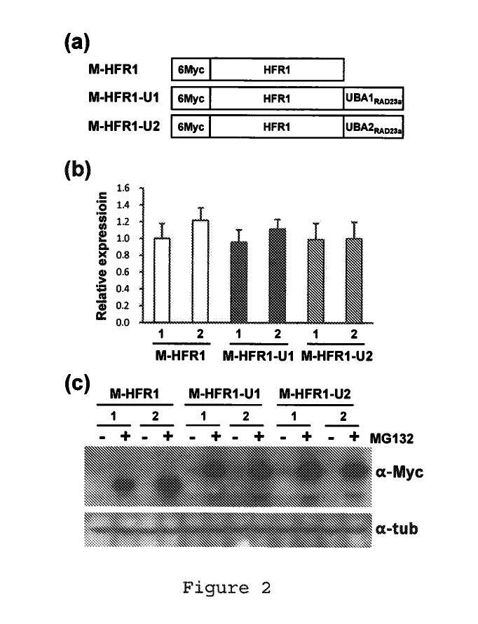

Members of the RAD23 protein family contain two UBA (Ub-associated domain) domains, one at the N-terminus (UBA1) and the other at the C-terminus (UBA2). Heessen et al. (2005) showed that UBA2 of the yeast RAD23 as well as the carboxyl terminal UBA of the human Ddi1 and Dsk2 (Heessen et al., 2005) when appended to an unstable reporter GFP can increase the stability of the latter. On the other hand, the yeast UBA1 was ineffective in the same assay. We decided to test Arabidopsis RAD23a-derived UBA1 and UBA2 for their capacity to prolong the half-life of unstable proteins (FIG. 2a).

Previous work has shown that the transcription factor HFR1 is an unstable protein even when expressed from a strong 35S promoter (Jang et al., 2005; Jang et al., 2007); the protein has a half life of about 0.5 hr and its overexpression in plants did not produce any morphological phenotype. To facilitate detection of HFR1 we fused 6× Myc tag to its N-terminus. We generated more than 20 independent transgenic lines and selected for further analysis 2 lines with comparable transcript levels as determined by RT-qPCR (FIG. 2b).

Using 6Myc-HFR1 as a control, we investigated the effects of UBA1 and UBA2 on HFR1 stability. The UBA1 or UBA2 was fused to the C-terminus of 6Myc-HFR1 (FIG. 2a) and more than 10 independent lines were obtained for each construct. We screened all transgenic lines by q-PCR and selected 2 lines each with transcript level comparable to those of the control 6Myc-HFR1 lines (FIG. 2b). We compared 6Myc-HFR1 protein levels in lines expressing 6Myc-HFR1, 6Myc-HFR-UBA1 and 6Myc-HFR1-UBA2 with and without MG132 treatment. FIG. 2c shows that whereas no protein was detected in the two 6Myc-HFR1 control lines, lines expressing not only 6Myc-HFR1-UBA2 but also 6Myc-HFR1-UBA1 displayed detectable HFR1 proteins. In all cases, HFR1 protein accumulation was highly increased upon inhibition of proteolysis by MG132.

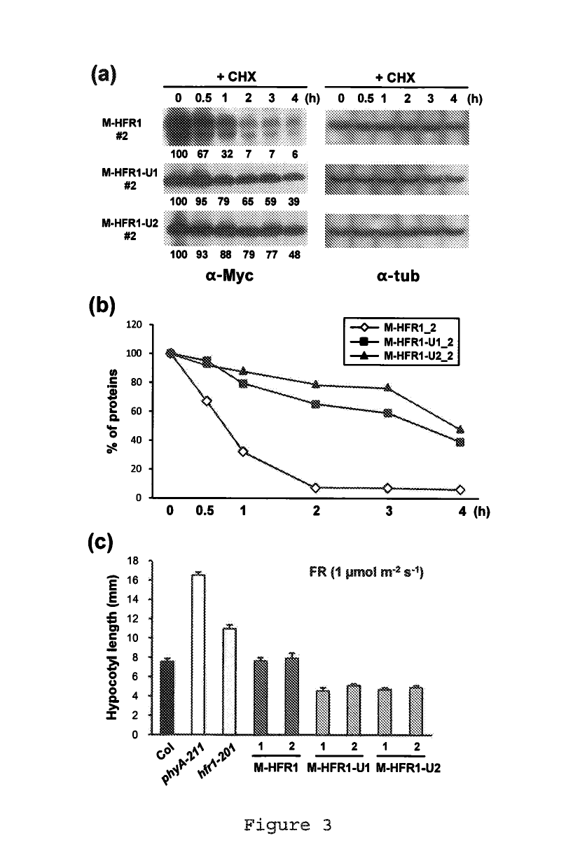

To assess the quantitative impact of UBA1/2 on 6Myc-HFR1 stability, we determined the protein half-life of various fusions in a cycloheximide chase experiments. Seedlings were first treated with MG132 to accumulate 6Myc-HFR1 proteins. After washing out of the proteasomal inhibitor MG132, cycloheximide was added to block new protein synthesis and changes in the various HFR1 fusion protein level were monitored in a time course experiment. FIGS. 3a and 3b show that 6Myc-HFR1 decayed rapidly with a half-life of about 0.75 hr consistent with previous data (Jang et al., 2005; Jang et al., 2007). Fusion of UBA2 prolonged the half-life by about 4-5 times. Surprisingly, similar results were also obtained with UBA1 (FIGS. 3a and 3b). These results indicate that UBA2 increased 6Myc-HFR1 stability and can be used to increase the stability of unstable proteins in plants. In contrast to yeast UBA1, the Arabidopsis UBA1 has similar properties as TJBA2, and thus can be used to increase the stability of unstable proteins in plants. In addition, because of the high amino acid sequence homology among the Arabidopsis RAD23 proteins, it is expected that the UBA1 and UBA2 domains of the RAD23b, RAD23c and RAD23d proteins will have similar properties as the UBA1 and UBA2 domains of RAD23a. Thus, these UBA domains can be used to increase the stability of unstable proteins in plants.

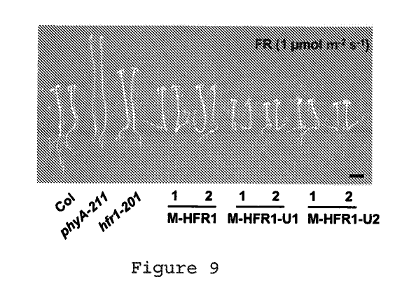

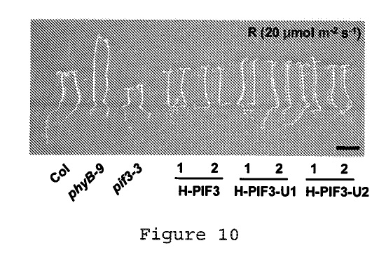

Although appending UBA1/2 could increase the HFR1 stability eventually the UBA-fusion proteins were also degraded. An important consideration is whether increased stability of a target protein would have any functional consequences, and if so, whether expression of the fusion protein would lead to an enhanced phenotype. To address these issues we investigated light sensitivity of HFR1 transgenic plants. FIGS. 3c and 9 show that overexpression of 6Myc-HFR1 had little effect on its FR sensitivity with respect to hypocotyl elongation; this is not surprising since this transcription factor is very unstable. By contrast, expression of 6Myc-HFR1-UBA2 clearly conferred FR hypersensitivity consistent with its higher accumulation levels. Similar results were obtained for 6Myc-HFR1-UBA1. Other than a FR hypersensitivity at the seedling stage, transgenic lines expressing 6-Myc-HFR1-UBA1 or 6-Myc-HFR1-UBA2 were phenotypically normal and fertile, and showed normal seed set.

### Example 4

### Increasing the Stability of PIF3

Next, we asked whether the RAD23 UBA1/2 can also be used to increase expression levels of other unstable proteins. A well-documented example of unstable proteins are the phytochrome-interacting factors (PIFs) which accumulate in darkness but are rapidly degraded in the light with a half-life of only 5-10 min (Al-Sady et al., 2006). Notwithstanding their instability, transgenic seedlings over-expressing PIFs are hyposensitive to light and have long hypocotyls because PIFs are negative regulators of photomorphogenesis.

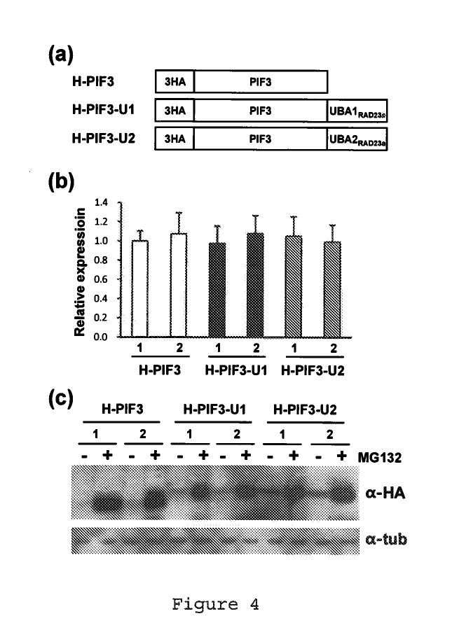

We selected PIF3 as a representative of this group of unstable proteins and used similar strategy as we had for HFR1 to fuse UBA1/2 to the C-terminus of 3HA-PIF3 (FIG. 4a). FIG. 4c shows that 3HA-PIF3 was barely detectable in light-grown seedlings but its expression level can be increased by the addition of MG132 indicating rapid proteolysis. Addition of UBA1/2 increased its stability such that 3-HA-PIF3-UBA2 and 3HA-PIF-UBA1 were detectable in untreated seedlings although MG132 treatment also elevated its accumulation levels. Note that these transgenic line had comparable transgene transcript levels (FIG. 4b).

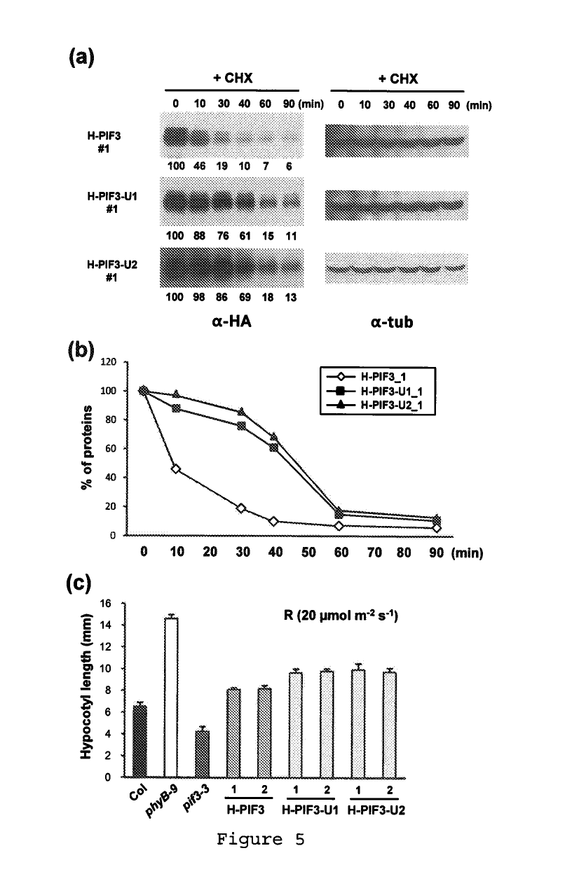

To obtain quantitative data, we treated dark-grown seedlings with MG132 overnight, washed out the inhibitor and then added cycloheximide to stop new protein synthesis. We then performed a time course experiments in the light to determine the decay rate of 3HA-PIF3 protein levels over a period of 90 min. FIGS. 5a and 5b show that in confirmation of previous results (Al-Sady et al., 2006) HA-PIF3 was rapidly degraded in the light with a half-life of approximately 10 min. Fusion of either UBA1 or UBA2 extended the half-life by about 4-5 times with no significant difference between the two fusions. Examination of seedlings grown under white light showed that 3HA-PIF3 conferred a hyposensitive phenotype with elongated hypocotyls and this phenotype was exaggerated in 3HA-PIF3-UBA1/2 (Figures Sc and 10). Adult plants of these transgenic lines did not show any abnormal morphology. The plants were fertile and produced seeds like WT plants.

### Example 5

### Increasing the Stability of JAZ10.1

Next, we examined whether UBA domain from other Arabidopsis proteins can be used to increase expression levels of unstable proteins. The Arabidopsis genome encodes an ortholog of yeast Ddi1 (DNA damage-inducible protein 1) and this protein contains one UBA domain in its C-terminus. We decided to use this UBA domain of the Arabidopsis DNA DAMAGE-INDUCIBLE 1 (DDI1).

With respect to target proteins we decided to investigate unstable signaling components involved in jasmonate signaling pathway. The JASMONATE ZIM-domain (JAZ) proteins are attractive candidates, since it has been shown that bioactive Jasmonates (JAs) promote interaction of JAZ proteins with CORONATINE INSENSITIVE1 (COI1), a component of the ubiquitin E3 ligase SCF_{COI1}, and this interaction leads to the ubiquitination and rapid degradation of JAZs by 26S proteasomes (Pauwels and Goossens, 2011). Among the 12 Arabidopsis genes encoding JAZ proteins we selected JAZ10.1 which encodes one of the three JAZ10 splice variants. Recent reports show that JAZ10.1-YFP signals was largely eliminated within 10 min of 50 μM MeJA treatment in 35S::JAZ10.1-YFP seedlings (Chung and Howe, 2009; Shyu et al., 2012).

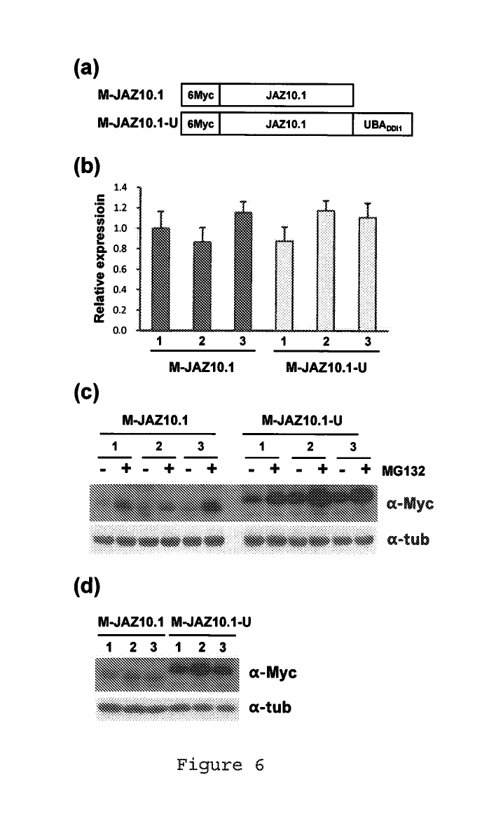

Using a similar strategy as has been used for HFR1 and PIF3, we generated more than 20 lines of each construct expressing 6Myc-JAZ10.1 or 6Myc-JAZ10.1-UBA (FIG. 6a) and selected 3 independent lines of each construct with comparable transcript levels for further analysis (FIG. 6b). FIG. 6c shows that 6Myc-JAZ10.1 was quite unstable even under normal condition (Chung and Howe, 2009) since its low expression levels can be elevated by MG132 treatment. Fusion of the Arabidopsis DDI1 UBA domain to C-terminal of JAZ10.1 considerable increased its expression levels in untreated seedlings, which can be slightly elevated by MG132 treatment (FIG. 6c). FIG. 6d shows a direct comparison of expression levels between 6Myc-JAZ10.1 and 6Myc-JAZ10.1-UBA lines without MG132 treatment.

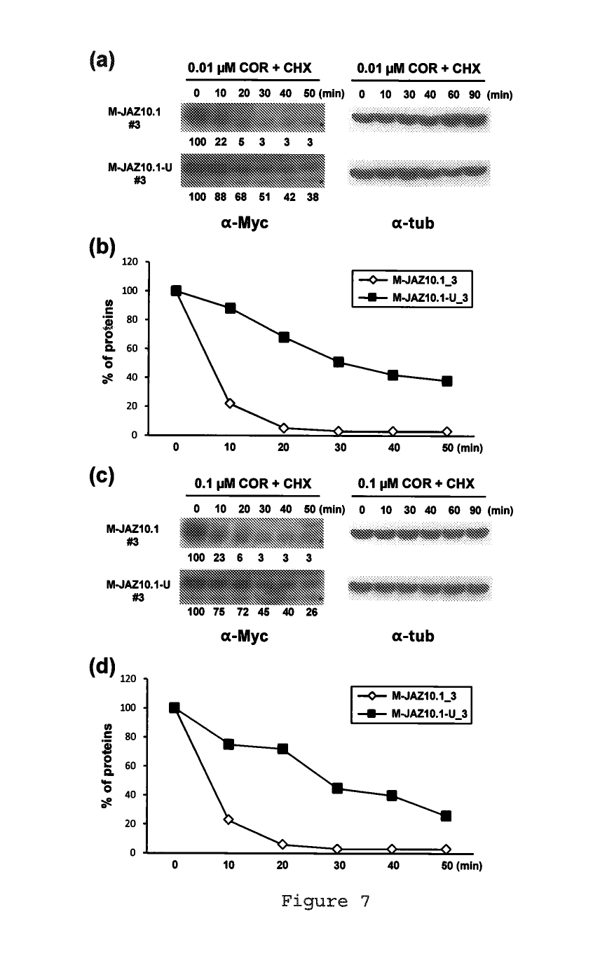

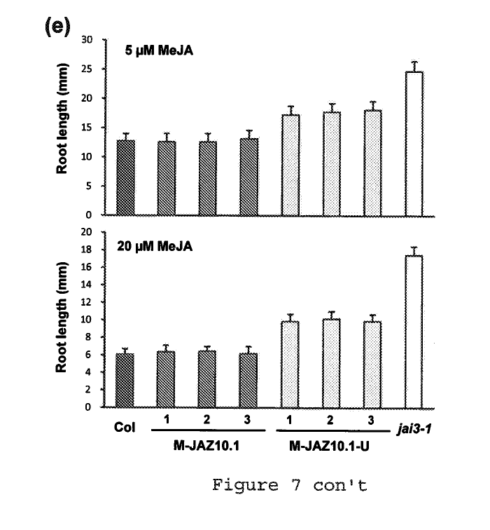

To assess the quantitative impact of the UBA domain on JAZ10.1 stability we performed time course experiments to compare the half-life of JAZ10.1 and its fusion protein. We treated transgenic seedlings with MG132 to maximize their expression levels. After 12 hr the proteasomal inhibitor was washed out and the decay rate of JAZ10.1 was measured after addition of cycloheximide plus the phytotoxin coronatine, a potent agonist of the COI-JAZ receptor system. FIGS. 7a and 7b show that upon 0.01 μM coronatine treatment 6Myc-JAZ10.1 was rapidly degraded with a half-life of approximately 5 min. Fusion of UBA extended the half-life of JAZ10.1 by about 6 times to about 30 min. Similar results were obtained with 0.1 μM coronatine confirming that 0.01 μM coronatine was already saturated for COI1-JAZ10.1 interaction in vivo. It should be noted that using ³H-labeled coronatine in saturation binding assays Shyu et al. (2012) showed that 7 nM coronatine was sufficient for COI1-JAZ10.1 interaction.

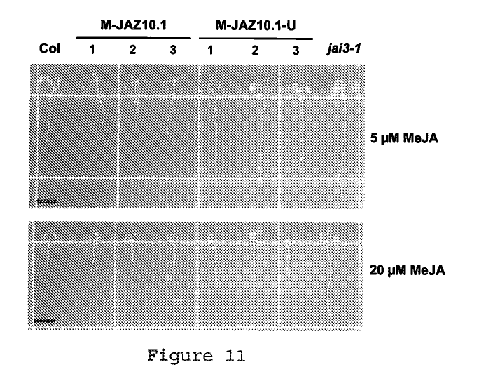

To determine whether UBA fusion would interfere with the biological activity of JAZ10.1 in JA responses, we performed JA-mediated root growth inhibition assays using WT (Col), jai3-1, 6Myc-JAZ10.1- and 6Myc-JAZ10.1-UBA-expressing lines. FIGS. 7e and 11 show that root growth of 6Myc-JAZ10.1 lines were as sensitive as WT (Col) seedlings to MeJA treatments (5 and 20 μM) as previously reported (Chung and Howe, 2009) whereas the root growth of 6Myc-JAZ10.1-UBA lines were less sensitive to MeJA treatments indicating retention of biological activity of the fusion protein. Note that we used WT (Col-0) and jar3-1 (Chini et al., 2007) as controls for JA-sensitive and JA-insensitive phenotypes in root growth inhibition assay under methyl jasmonate (MeJA) treatment.

Optimization of protein expression in transgenic plants is a subject of considerable interest for plant biotechnology. Previous work in this area has mainly dealt with increasing transcriptional output and enhancing translational efficiency (Streatfield, 2007). More recently, with the recognition that a subset of TF transcripts is subject to miRNA-mediated cleavage, efforts have been made to engineer cleavage-resistant transcripts leading to increased transcript and protein expression levels and enhanced phenotypic outcome (Guo et al., 2005). However, as far as we know, few attempts have been made to increase stability of transgene-derived proteins (Garbarino et al., 1995; Hondred et al., 1999). Overexpression of genes encoding TF and signaling pathway components (e.g., E3 ligases) has become a commonly used strategy to explore gene regulatory circuits or to introduce into crop plants a desirable agronomic trait (Shinozaki et al., 2003). The success of such experiments may depend on the stability of transgene-derived protein products.

Here, we have specifically addressed the issue of how to increase protein stability in transgenic plants. To avoid complications introduced by possible changes in transcript levels and/or translational efficiency during the course of experiment, we have used a previously developed method to determine protein decay rate in the absence of new protein synthesis pang et al., 2005; Jang et al., 2007). Previous experiments with stabilization of proteins in yeast were carried out with an engineered, destabilized GFP reporter protein (Heessen et al., 2005; Heinen et al., 2011). However, there is some evidence that results obtained from such artificial reporter proteins may not necessarily apply to physiological substrates (Verma et al., 2004). To avoid such complications, we have decided to choose two Arabidopsis TFs and one signal mediator that are highly unstable with a half-life of 5-40 min. HFR1 and PIF3 are positive and negative regulatory components of phytochrome signaling pathway, respectively, whereas JAZ10.1 is a negative regulator of jasmonate signaling. We show here that Arabidopsis RAD23 proteins are stable and the UBA2 from RAD23a can be used as a transferrable stabilizing signal to increase protein half-life of the two TFs by about 4-5 times. Similar results were obtained with the UBA domain of the Arabidopsis DDI1 protein using the unstable JAZ10.1 as a reporter protein. Although the increase in TF protein half-life was not overly dramatic, the improved stability of the TF-UBA2 fusion proteins was sufficient to give an enhanced phenotype compared to that obtained with the control reporter protein alone. These results also indicate that the reporter-UBA2 fusion proteins are biologically active. We note that a moderate increase in reporter protein stability is probably preferable as a huge increase in expression levels of reporter proteins, especially if they are signaling components, may lead to growth defects in expressing transgenic plants. Our results on the Arabidopsis UBA2 function are similar to those reported in yeast in which the yeast UBA2 has been reported to inhibit rapid degradation of an unstable GFP protein (Heessen et al., 2005). Taken together, the results suggest that the function of UBA2 is conserved during evolution.

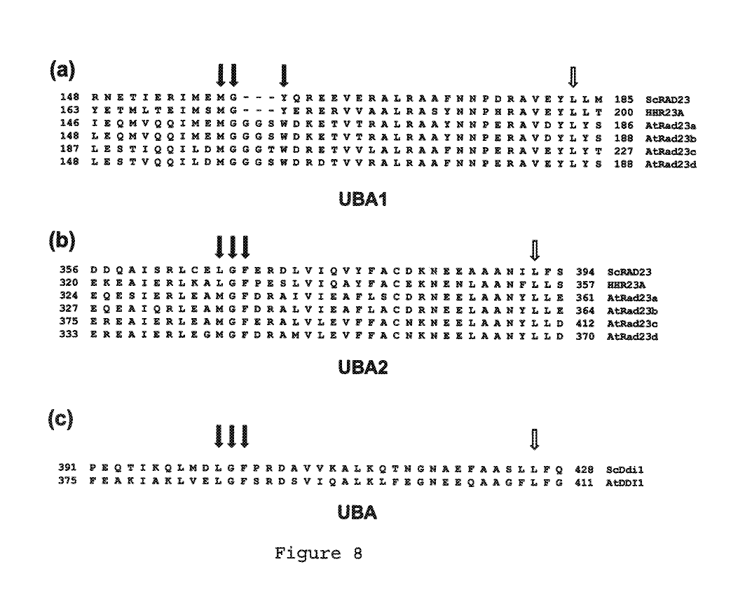

Surprisingly, we found that in contrast to yeast (Heessen et al., 2005), the Arabidopsis RAD23a UBA1 has similar activity as UBA2. No differences could be detected between the two UBA domains with respect to their capacity to prolong protein half-life with consequential phenotypic impact. Sequence comparison shows 52.6% amino acid identity between yeast UBA1 and Arabidopsis UBA1 (RAD23a); the comparable number for UBA2 is 44.7%. UBA1 of HHR23A (human homologue of yeast RAD23A) shares low amino acid sequence homology but high structural similarity with a conserved large hydrophobic surface patch with UBA2 (Mueller and Feigon, 2002). Analysis of mutant derivatives of UBA domain confirmed the requirement of several key amino acids including Met-Gly-Phe/Tyr (MGF/Y loop) and Leu335 for ubiquitin binding (Bertolaet et al., 2001; Mueller and Feigon, 2002; Ohno et al., 2005). In addition, a single L392A amino acid substitution abrogates the protective effect of the UBA2 domain in yeast (Heessen et al., 2005). FIG. 8 compares amino acid sequences of Arabidopsis UBA1/2 domains with those of yeast and human RAD23. Similar to yeast and human RAD23 we found comparable MGF/Y loops and Leu residues in the UBA2 domain of Arabidopsis RAD23a. In contrast to the UBA1 in yeast and human RAD23, however, the Arabidopsis UBA1 domains contain a 3-amino acid insertion (Gly-Gly-Ser/Thr) between Met-Gly and an aromatic amino acid (Y/W). This structural difference might explain the possible activity of Arabidopsis UBA1 domain in prolonging protein half-life. Indeed, substitution of a tyrosine residue in MGY motif in the UBA 1 domain with a phenylalanine residue significantly increased the steady state levels of a destabilized GFP reporter protein in yeast (Heinen et al., 2011).

How UBA domains stabilize the destabilized GFP protein in yeast is not entirely clear. An attractive hypothesis is that the C-terminally located UBA dfomain blocks or delays unfolding of proteasome-bound fusion proteins. This hypothesis is consistent with the observation that the UBA2 protective effect is abolished when its structural integrity is disrupted (Heesseen et al., 2005) or C-terminal unstructured polypeptides is added (Heinen et al., 2011). Indeed, RAD23 itself lacks an effective initiation region for proteasomes to recognize and unfold thus resulting in relative stability. If this is the case, then the Arabidopsis UBA1 should have a similar function. Irrespective of the mechanism, the Arabidopsis UBA1/2 domains can be used as portable cis-acting stabilizing signals to prolong half-life of any unstable proteins (e.g. E3 ligases) located in cyotosol or nuclei of plant cells.

As shown herein, UBA1/2 from the Arabidopsis RAD23a and UBA from the Arabidopsis DDI1 proteins were able to increase the stability of two plant transcription factors and one signaling component protein, respectively. We found that all UBAs are effective in prolonging the half-life of HFR1 (Fairchild et al., 2000), PIF3 (Ni et al., 1993) and JAZ10.1 (Chung and Howe, 2009) and the fusion proteins retained biological activities. The results described herein demonstrate that UBA fusions can be used to increase stability of unstable proteins which is useful for crop improvement.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

Al-Sady, B., Ni, W., Kircher, S., Schäfer, E. and Quail, P. H. (2006). Photoactivated phytochrome induces rapid PIF3 phosphorylation prior to proteasome-mediated degradation. Mol Cell 23, 439-446.
Bertolaet, B. L., Clarke, D. J., Wolff, M., Watson, M. H., Henze, M., Divita, G. and Reed, S. I. (2001). UBA domains of DNA damage-inducible proteins interact with ubiquitin. Nat Struct Mol Biol 8, 417-422.
Chini, A., Fonseca, S., Fernández, G., Adie, B., Chico, J. M., Lorenzo, O., García-Casado, G., López-Vidriero, I., Lozano, F. M., Ponce, M. R., Micol, J. L. and Solano R. (2007). The JAZ family of repressors is the missing link in jasmonate signalling. Nature 448: 666-671.
Chiu, W. et al. (1996). Engineered GFP as a vital reporter in plants. Current Biology 6:325-330.
Christensen, A. H. and Quail, P. H, (1989). Sequence analysis and transcriptional regulation by heat shock of polyubiquitin transcripts from maize. Plant Mol Biol 12:619-632.
Christensen, A. H. et al. (1992). Maize polyubiquitin genes: structure, thermal perturbation of expression and transcript splicing, and promoter activity following transfer to protoplasts by electroporation. Plant Mol Biol 18:675-689.
Chung, H. S. and Howe, G. A. (2009). A critical role for the TIFY motif in repression of jasmonate signaling by a stabilized splice variant of the JASMONATE ZIM-domain protein JAZ10 in Arabidopsis. Plant Cell 21, 131-145.
De Wet, J. R. et al. (1987). Firefly luciferase gene: structure and expression in mammalian cells. Mol Cell Biol 7:725-737.
Fairchild, C. D., Schumaker, M. A. and Quail, P. H. (2000). HFR1 encodes an atypical bHLH protein that acts in phytochrome A signal transduction. Genes Dev 14, 2377-2391.
Farmer, L. M., Book, A. J., Lee, K. H., Lin, Y. L., Fu, H. and Vierstra, R. D. (2010). The RAD23 family provides an essential connection between the 26S proteasome and ubiquitylated proteins in Arabidopsis. Plant Cell 22, 124-142.
Fu, H., Lin, Y. L. and Fatimababy, A. S. (2010.) Proteasomal recognition of ubiquitylated substrates. Trends Plant Sci 15, 375-386.
Garbarino, J. E., Oosumi, T. and Belknap, W. R. (1995). Isolation of a polyubiquitin promoter and its expression in transgenic potato plants. Plant Physiol 109, 1371-1378.
Goff, S. A. et al. (1990). Transactivation of anthocyanin biosynthetic genes following transfer of B regulatory genes into maize tissues. EMBO J 9:2517-2522.
Guo, H. S., Xie, Q., Fei, J. F. and Chua, N. H. (2005). MicroRNA directs mRNA cleavage of the transcription factor NAC1 to downregulate auxin signals for arabidopsis lateral root development. Plant Cell 17, 1376-1386.
Heessen, S., Dantuma, N. P., Tessarz, P., Jellne, M. and Masucci, M. G. (2003). Inhibition of ubiquitin/proteasome-dependent proteolysis in Saccharomyces cerevisiae by a Gly-Ala repeat. FEBS Letters 555, 397-404.
Heessen, S., Masucci, M. G. and Dantuma, N. P. (2005). The UBA2 domain functions as an intrinsic stabilization signal that protects Rad23 from proteasomal degradation. Mol Cell 18, 225-235.
Heinen, C., Acs, K., Hoogstraten, D. and Dantuma, N. P. (2011). C-terminal UBA domains protect ubiquitin receptors by preventing initiation of protein degradation. Nat Commun 2, 1-9.
Henriques, R., Jang, L C. and Chua, N. H. (2009). Regulated proteolysis in light-related signaling pathways. Curr Opin Plant Biol 12, 49-56.
Hondred, D., Walker, J. M., Mathews, D. E. and Vierstra, R. D. (1999). Use of ubiquitin fusions to augment protein expression in transgenic plants. Plant Physiol 119, 713-724.
James, E. and Lee, J. M. (2001). The production of foreign proteins from genetically modified plant cells. Adv Biochem Eng/Biotechnol 72, 127-155.
Jang, I. C., Yang, J. Y., Seo, H. S. and Chua, N. H. (2005). HFR1 is targeted by COP1 E3 ligase for post-translational proteolysis during phytochrome A signaling. Genes Dev 19, 593-602.
Jang, I. C., Yang, S. W., Yang, J. Y. and Chua, N. H. (2007). Independent and interdependent functions of LAF1 and HFR1 in phytochrome A signaling. Genes Dev 21, 2100-2111
Jefferson, R. A. et al. (1991). Plant Molecular Biology Manual, ed. Gelvin et al., Kluwer Academic Publishers, pp. 1-33.
Kain, S. R. et al. (1995). Green fluorescent protein as a reporter of gene expression and protein localization. BioTechniques 19, 650-655.
Last, D. I. et al. (1991). pEmu: an improved promoter for gene expression in cereal cells. Theor Appl Genet 81:581-588.
LaVallie, E. R., McCoy, J. M., Smith, D. B. and Riggs, P. (2001). Enzymatic and Chemical Cleavage of Fusion Proteins. Current Protocols in Molecular Biology 28:16.4.5-16-16.4.17.
Mallory, A. and Vaucheret, H. (2010). Form, Function, and Regulation of ARGONAUTE Proteins. Plant Cell 22, 3879-3889.
McElroy, D. et al. (1990). Isolation of an efficient actin promoter for use in rice transformation. Plant Cell 2:163-171.
Mitsuda, N. and Ohme-Takagi, M. (2009). Functional analysis of transcription factors in Arabidopsis. Plant Cell Physiol 50, 1232-1248.
Mueller, T. D. and Feigon, J. (2002). Solution structures of UBA domains reveal a conserved hydrophobic surface for protein-protein interactions. J Mol Biol 319, 1243-1255.
Ni, M., Tepperman, J. M. and Quail, P. H. (1998). PIF3, a phytochrome-interacting factor necessary for normal photoinduced signal transduction, is a novel basic helix-loop-helix protein. Cell 95, 657-667.
Odell, J. T. et al. (1985). Identification of DNA sequences required for activity of the cauliflower mosaic virus 35S promoter. Nature 313:810-812.
Ohno, A., Jee, J., Fujiwara, K., Tenno, T., Goda, N., Tochio, H., Kobayashi, H., Hiroaki, H. and Shirakawa, M. (2005). Structure of the UBA domain of Dsk2p in complex with ubiquitin molecular determinants for ubiquitin recognition. Structure 13, 521-532.
Seo, H. S., Yang, J. Y., Ishikawa, M., Bolle, C., Ballesteros, M. L. and Chua, N. H. (2003). LAF1 ubiquitination by COP1 controls photomorphogenesis and is stimulated by SPA1. Nature 423, 995-999.
Shinozaki, K., Yamaguchi-Shinozaki, K. and Seki, M. (2003). Regulatory network of gene expression in the drought and cold stress responses. Curr Opin Plant Biol 6, 410-417.
Shyu, C., Figueroa, P., Depew, C. L., Cooke, T. F., Sheard, L. B., Moreno, J. E, Katsir, L., Zheng, N., Browse, J., Howe, G. A. (2012). JAZ8 Lacks a Canonical Degron and Has an EAR Motif That Mediates Transcriptional Repression of Jasmonate Responses in Arabidopsis. Plant Cell [Epub Feb. 10, 2012 ahead of print: http://dx.doi.org/10.1105/tpc.111.093005].
Streatfield, S. J. (2007). Approaches to achieve high-level heterologous protein production in plants. Plant Biotechnol J5, 2-15.
Velten, J. et al. (1984). Isolation of a dual plant promoter fragment from the Ti plasmid of Agrobacterium tumefaciens. EMBO J3, 2723-2730.
Vierstra, R. D. (2009). The ubiquitin-26S proteasome system at the nexus of plant biology. Nat Rev Mol Cell Biol 10, 385-397.
Voges, D., Zwickl, P. and Baumeister, W. (1999). The 26S proteasome: a molecular machine designed for controlled proteolysis. Annu Rev Biochem 68, 1015-1068.
Xie, Q., Frugis, G., Colgan, D. and Chua, N. H. (2001). Arabidopsis NAC1 transduces auxin signal downstream of TIR1 to promote lateral root development. Genes Dev 14, 3024-3036.
Xu, J. and Chua, N. H. (2011). Processing bodies and plant development. Curr Opin Plant Biol 14, 88-93.
Yang, J. et al. (2003). Intein-mediated assembly of a functional β-glucuronidase in transgenic plants. Proc Natl Acad Sci USA 100, 3513-3518.

1. A nucleic acid construct comprising a plant operable promoter operably linked to a nucleic acid encoding a fusion protein, wherein the fusion protein comprises a first protein segment and a second protein segment, wherein the first protein segment is a protein of interest and the second protein segment is a UBA domain of a protein, wherein the second protein segment is fused to the C-terminus of the first protein segment and wherein the UBA domain stabilizes the protein of interest when the fusion protein is expressed in a plant cell.

2. The nucleic acid construct of claim 1, wherein the nucleic acid comprises a first nucleic acid segment encoding the protein of interest and a second nucleic acid segment encoding the UBA domain.

3. The nucleic acid construct of claim 1, wherein the UBA domain is selected from the group consisting of:
(a) a UBA1 domain of an Arabidopsis RAD23 protein;
(b) a UBA2 domain of an Arabidopsis RAD23 protein;
(c) a UBA2 domain of a yeast RAD23 protein;
(d) a UBA domain of an Arabidopsis DDI1 protein;
(e) a UBA domain of a yeast Ddi1 protein;
(f) a UBA domain of an Arabidopsis UBL1 protein;
(g) a UBA domain of an Arabidopsis DSK2a protein;
(i) a UBA domain of an Arabidopsis DSK2b protein;
(j) a UBA1 domain of an Arabidopsis NUB1 protein; and
(k) a UBA2 domain of an Arabidopsis NUB1 protein.

4. The nucleic acid construct of claim 3, wherein the Arabidopsis RAD23 protein is selected from the group consisting of Arabidopsis RAD23a protein, Arabidopsis RAD23b protein, Arabidopsis RAD23c protein and Arabidopsis RAD23d protein.

5. The nucleic acid construct of claim 1, wherein the second protein segment is a UBA domain selected from the group consisting of a native UBA domain and a conservative variant of the native UBA domain.

6. The nucleic acid construct of claim 5, wherein the UBA domain is a native UBA1 domain of an Arabidopsis RAD23 protein or a conserved variant of the native UBA1 domain of the Arabidopsis RAD23 protein, wherein the native UBA1 domain has an amino acid sequence selected from the group consisting of the amino acid sequence set forth in SEQ ID NO:1, the amino acid sequence set forth in SEQ ID NO:9, the amino acid sequence set forth in SEQ ID NO:10, the amino acid sequence set forth in SEQ ID NO:11, and the amino acid sequence set forth in SEQ ID NO:12.

7. The nucleic acid construct of claim 5, wherein the UBA domain is a native UBA2 domain of an Arabidopsis RAD23 protein or a conserved variant of the native UBA2 domain of the Arabidopsis RAD23 protein, wherein the native UBA2 domain has an amino acid sequence selected from the group consisting of the amino acid sequence set forth in SEQ ID NO:2, the amino acid sequence set forth in SEQ ID NO:15, the amino acid sequence set forth in SEQ ID NO:16, the amino acid sequence set forth in SEQ ID NO:17, and the amino acid sequence set forth in SEQ ID NO:18.

8. The nucleic acid construct of claim 5, wherein the UBA domain is a native UBA domain or a conserved variant of the native UBA domain, wherein the native UBA domain has an amino acid sequence selected from the group consisting of the amino acid sequence set forth in SEQ ID NO:13, the amino acid sequence set forth in SEQ ID NO:19, the amino acid sequence set forth in SEQ ID NO:20, the amino acid sequence set forth in SEQ ID NO:21, the amino acid sequence set forth in SEQ ID NO:22, the amino acid sequence set forth in SEQ ID NO:23, the amino acid sequence set forth in SEQ ID NO:24, the amino acid sequence set forth in SEQ ID NO:25, the amino acid sequence set forth in SEQ ID NO:26, the amino acid sequence set forth in SEQ ID NO:27, the amino acid sequence set forth in SEQ ID NO:28, the amino acid sequence set forth in SEQ ID NO:29, the amino acid sequence set forth in SEQ ID NO:30, the amino acid sequence set forth in SEQ ID NO:31, and the amino acid sequence set forth in SEQ ID NO:32.

9. The nucleic acid construct of claim 1, wherein the protein of interest is a protein to be expressed in a transgenic plant cell or a transgenic plant.

10. A fusion protein encoded by the nucleic acid construct of claim 1.

11. A transgenic plant cell, a transgenic plant or a transgenic seed comprising the nucleic acid construct of claim 1.

12. The transgenic plant cell, transgenic plant or transgenic seed of claim 11, wherein the fusion protein is expressed in the transgenic plant cell, the transgenic plant or the transgenic plant seed.

13. The transgenic plant of claim 12, wherein the expressed fusion protein imparts a phenotypic characteristic to the transgenic plant.

14. A method of enhancing the stability of an expressed protein in a transgenic plant cell or a transgenic plant which comprises:
(a) transfecting a plant cell with the nucleic acid construct of claim 1 to produce a transgenic plant cell having the nucleic acid construct;
(a1) optionally regenerating a transgenic plant from the transgenic plant cell; and
(b) expressing the fusion protein in the transgenic plant cell or the transgenic plant,
whereby the expressed fusion protein is more stable in the transgenic plant cell or the transgenic plant than the corresponding protein of interest and has the same function in the transgenic plant cell or the transgenic plant as the corresponding protein of interest.

15. The nucleic acid construct of claim 2, wherein the UBA domain is selected from the group consisting of:
(a) a UBA1 domain of an Arabidopsis RAD23 protein;
(b) a UBA2 domain of an Arabidopsis RAD23 protein;
(c) a UBA2 domain of a yeast RAD23 protein;
(d) a UBA domain of an Arabidopsis DDI1 protein;
(e) a UBA domain of a yeast Ddi1 protein;
(f) a UBA domain of an Arabidopsis UBL1 protein;
(g) a UBA domain of an Arabidopsis DSK2a protein;
(i) a UBA domain of an Arabidopsis DSK2b protein;
(j) a UBA1 domain of an Arabidopsis NUB1 protein; and
(k) a UBA2 domain of an Arabidopsis NUB1 protein.

16. The nucleic acid construct of claim 15, wherein the Arabidopsis RAD23 protein is selected from the group consisting of Arabidopsis RAD23a protein, Arabidopsis RAD23b protein, Arabidopsis RAD23c protein and Arabidopsis RAD23d protein.

17. The nucleic acid construct of claim 2, wherein the second protein segment is a UBA domain selected from the group consisting of a native UBA domain and a conservative variant of the native UBA domain.

18. The nucleic acid construct of claim 17, wherein the UBA domain is a native UBA1 domain of an Arabidopsis RAD23 protein or a conserved variant of the native UBA1 domain of the Arabidopsis RAD23 protein, wherein the native UBA1 domain has an amino acid sequence selected from the group consisting of the amino acid sequence set forth in SEQ ID NO:1, the amino acid sequence set forth in SEQ ID NO:9, the amino acid sequence set forth in SEQ ID NO:10, the amino acid sequence set forth in SEQ ID NO:11, and the amino acid sequence set forth in SEQ ID NO:12.

19. The nucleic acid construct of claim 17, wherein the UBA domain is a native UBA2 domain of an Arabidopsis RAD23 protein or a conserved variant of the native UBA2 domain of the Arabidopsis RAD23 protein, wherein the native UBA2 domain has an amino acid sequence selected from the group consisting of the amino acid sequence set forth in SEQ ID NO:2, the amino acid sequence set forth in SEQ ID NO:15, the amino acid sequence set forth in SEQ ID NO:16, the amino acid sequence set forth in SEQ ID NO:17, and the amino acid sequence set forth in SEQ ID NO:18.

20. The nucleic acid construct of claim 17, wherein the UBA domain is a native UBA domain or a conserved variant of the native UBA domain, wherein the native UBA domain has an amino acid sequence selected from the group consisting of the amino acid sequence set forth in SEQ ID NO:13, the amino acid sequence set forth in SEQ ID NO:19, the amino acid sequence set forth in SEQ ID NO:20, the amino acid sequence set forth in SEQ ID NO:21, the amino acid sequence set forth in SEQ ID NO:22, the amino acid sequence set forth in SEQ ID NO:23, the amino acid sequence set forth in SEQ ID NO:24, the amino acid sequence set forth in SEQ ID NO:25, the amino acid sequence set forth in SEQ ID NO:26, the amino acid sequence set forth in SEQ ID NO:27, the amino acid sequence set forth in SEQ ID NO:28, the amino acid sequence set forth in SEQ ID NO:29, the amino acid sequence set forth in SEQ ID NO:30, the amino acid sequence set forth in SEQ ID NO:31, and the amino acid sequence set forth in SEQ ID NO:32.

21. A fusion protein encoded by the nucleic acid construct of claim 3.

22. A fusion protein encoded by the nucleic acid construct of claim 5.

23. A transgenic plant cell, a transgenic plant or a transgenic seed comprising the nucleic acid construct of claim 3.

24. A transgenic plant cell, a transgenic plant or a transgenic seed comprising the nucleic acid construct of claim 5.

25. A method of enhancing the stability of an expressed protein in a transgenic plant cell or a transgenic plant which comprises:
(a) transfecting a plant cell with the nucleic acid construct of claim 3 to produce a transgenic plant cell having the nucleic acid construct;
(a1) optionally regenerating a transgenic plant from the transgenic plant cell; and
(b) expressing the fusion protein in the transgenic plant cell or the transgenic plant,
whereby the expressed fusion protein is more stable in the transgenic plant cell or the transgenic plant than the corresponding protein of interest and has the same function in the transgenic plant cell or the transgenic plant as the corresponding protein of interest.

26. A method of enhancing the stability of an expressed protein in a transgenic plant cell or a transgenic plant which comprises:
(a) transfecting a plant cell with the nucleic acid construct of claim 5 to produce a transgenic plant cell having the nucleic acid construct;
(a1) optionally regenerating a transgenic plant from the transgenic plant cell; and
(b) expressing the fusion protein in the transgenic plant cell or the transgenic plant,
whereby the expressed fusion protein is more stable in the transgenic plant cell or the transgenic plant than the corresponding protein of interest and has the same function in the transgenic plant cell or the transgenic plant as the corresponding protein of interest.


SEQUENCE LISTING (34 sequences)

SEQ ID NO: 1 - protein, 44 aa, Arabidopsis thaliana
GSSIEQMVQQIMEMGGGSWDKETVTRALRAAYNNPERAVDYLYS

SEQ ID NO: 2 - protein, 39 aa, Arabidopsis thaliana
EEQESIERLEAMGFDRAIVIEAFLSCDRNEELAANYLLE

SEQ ID NO: 3 - DNA, 20 bp, Arabidopsis thaliana
gaaacgacgtcggatcactt

SEQ ID NO: 4 - DNA, 20 bp, Arabidopsis thaliana
ttcatcagggacaacaacca

SEQ ID NO: 5 - DNA, 20 bp, Arabidopsis thaliana
agcttttcaggctcaccaaa

SEQ ID NO: 6 - DNA, 20 bp, Arabidopsis thaliana
gcttgtggtggaggaatgtt

SEQ ID NO: 7 - protein, 38 aa, Saccharomyces cerevisiae
RNETIERIMEMGYQREEVERALRAAFNNPDRAVEYLLM

SEQ ID NO: 8 - protein, 38 aa, Homo sapiens
YETMLTEIMSMGYERERVVAALRASYNNPHRAVEYLLT

SEQ ID NO: 9 - protein, 41 aa, Arabidopsis thaliana
IEQMVQQIMEMGGGSWDKETVTRALRAAYNNPERAVDYLYS

SEQ ID NO: 10 - protein, 41 aa, Arabidopsis thaliana
LEQMVQQIMEMGGGSWDKETVTRALRAAYNNPERAVDYLYS

SEQ ID NO: 11 - protein, 41 aa, Arabidopsis thaliana
LESTIQQILDMGGGTWDRETVVLALRAAFNNPERAVEYLYT

SEQ ID NO: 12 - protein, 41 aa, Arabidopsis thaliana
LESTVQQILDMGGGSWDRDTVVRALRAAFNNPERAVEYLYS

SEQ ID NO: 13 - protein, 38 aa, Saccharomyces cerevisiae
DDQAISRLCELGFERDLVIQVYFACDKNEEAAANILFS

SEQ ID NO: 14 - protein, 38 aa, Homo sapiens
EKEAIERLKALGFPESLVIQAYFACEKNENLAANFLLS

SEQ ID NO: 15 - protein, 38 aa, Arabidopsis thaliana
EQESIERLEAMGFDRAIVIEAFLSCDRNEELAANYLLE

SEQ ID NO: 16 - protein, 38 aa, Arabidopsis thaliana
EQEAIQRLEAMGFDRALVIEAFLACDRNEELAANYLLE

SEQ ID NO: 17 - protein, 38 aa, Arabidopsis thaliana
EREAIERLEAMGFERALVLEVFFACNKNEELAANYLLD

SEQ ID NO: 18 - protein, 38 aa, Arabidopsis thaliana
EREAIERLEGMGFDRAMVLEVFFACNKNEELAANYLLD

SEQ ID NO: 19 - protein, 38 aa, Arabidopsis thaliana
DDEAINRLEAMGFERRVVLEVFLACNKNEQLAANFLLD

SEQ ID NO: 20 - protein, 38 aa, Arabidopsis thaliana
FEAKIAKLVELGFSRDSVIQALKLFEGNEEQAAGFLFG

SEQ ID NO: 21 - protein, 39 aa, Arabidopsis thaliana
FATQLQQLQEMGFYDRAENIRALLATNGNVNAAVERLLG

SEQ ID NO: 22 - protein, 39 aa, Arabidopsis thaliana
YATQLQQLQEMGFYDRAENIRALLATNGNVNAAVERLLG

SEQ ID NO: 23 - protein, 38 aa, Arabidopsis thaliana
PDETLSLVMGMGFQEKDAKRALRLNNQDIASSVDFLIE

SEQ ID NO: 24 - protein, 38 aa, Arabidopsis thaliana
DMQMLERLVSIGYARELAAESLRRNENDIQKALDILTD

SEQ ID NO: 25 - protein, 38 aa, Saccharomyces cerevisiae
PEQTIKQLMDLGFPRDAVVKALKQTNGNAEFAASLLFQ

SEQ ID NO: 26 - protein, 38 aa, Populus trichocarpa
PDEALSLVMSMGFGEWDAKRALRMSNQDIQSAVNFLVV

SEQ ID NO: 27 - protein, 38 aa, Populus trichocarpa
DLQRLTEVVSIGFEKELAAEALRKNENDTQKALDDLTN

SEQ ID NO: 28 - protein, 38 aa, Ricinus communis
PDEALSIVMGMGFKENDAKRALRMSNQDIESAINFLVE

SEQ ID NO: 29 - protein, 38 aa, Ricinus communis
DLQRLKELVSLGFEKELAAEALRRNENDSEKALDDLTN

SEQ ID NO: 30 - protein, 38 aa, Vitis vinifera
DLQSLHMLVSIGFEKELAAEALRRNENDTQKALDDLTN

SEQ ID NO: 31 - protein, 38 aa, Oryza sativa
NMQKLKGLVAIGFEKKLAAEALRINENDADKALDLLTD

SEQ ID NO: 32 - protein, 38 aa, Sorghum bicolor
DMQKLKGLTAIGFEKYLAAEALRINENDADKALDLLTN

SEQ ID NO: 33 - DNA, 20 bp, Arabidopsis thaliana
tcggtaattcttccgaccac

SEQ ID NO: 34 - DNA, 20 bp, Arabidopsis thaliana
gccgatgtcggatagtaagg